(19) United States
(12) Patent Application Publication
(10) Pub. No.: US 2026/0345938 A1
(43) Pub. Date: Nov. 5, 2026

(54) PATIENT INTERFACE

(71) Applicant: Fisher & Paykel Healthcare Limited, Auckland (NZ)

(72) Inventors: Benjamin John TAIT, Auckland (NZ); Andrew James HILLIARD, Auckland (NZ); Madeline Rose PARKER, Auckland (NZ)

(21) Appl. No.: 19/141,620

(22) PCT Filed: Dec. 22, 2023
(86) PCT No.: PCT/IB2023/063146

§ 371 Date: Jun. 20, 2025

Related U.S. Application Data
(60) Provisional application No. 63/476,903, filed on Dec. 22, 2022.

(51) Int. Cl. A61M 16/06; A61M 25/00; A61M 29/00

(52) U.S. Cl. CPC A61M 16/0666; A61M 29/00; A61M 2025/006; A61M 2240/00

(57) ABSTRACT

A prong for a nasal cannula, wherein the prong comprises a proximal end, a distal end, and inner surface and an outer surface. The proximal end has an opening configured to deliver a gas into a naris. The distal end configured to be in fluid communication with a gas source. An inner surface of the prong defines a passage between the proximal end and the distal. At least one protrusion is provided on the outer surface of the prong. The at least one protrusion provides an increased resistance to a flow of exhaled gas along the outer surface of the prong. Patient interfaces and systems for delivery of a breathable gas, each comprising a prong with at least one protrusion, are also provided.

### TECHNICAL FIELD

The present disclosure generally relates to patient interfaces for providing breathable gas flow to a patient. More particularly, the present disclosure relates to patient interfaces of the type having one or more prongs for delivering a gas to the nare(s) of a person, the prong(s) having one or more protrusions on its outer surface.

### BACKGROUND

Assisted breathing systems are available to aid patients in breathing for a number of reasons, for example due to, or in recovery from, a medical condition, during or following a medical procedure or otherwise for individuals who require a form of breathing support. In assisted breathing, respiratory gases are supplied to a patient through a flexible breathing tube. The gases expired by the patient may be channelled through a similar breathing tube or expelled to the patient's surroundings. The gases are typically administered to the patient through a patient interface, which may also comprise a short length of dedicated breathing tube to couple the interface with the supply tube. The patient interface may receive breathing gas from the flexible breathing tube and deliver it to the patient. Examples of a patient interface include a nasal cannula, nasal mask, oronasal or full face mask, and endotracheal (ET) tube.

Nasal high flow (NHF) is a form of respiratory support typically having an open system that provides breathable gas to a patient via a patient interface. The patient interface has prongs that are configured for insertion into the nares of the patient. The system is open because the prongs do not form an airtight seal with the nares of the patient and a gas exhaled by the patient can exit the nares in the space between the outer surface of the prongs and the inner surface of the nares.

It would be desirable to overcome one or more problem associated with the prior art and/or it would be desirable to provide the user of a useful choice.

### SUMMARY

A nasal cannula or interface system typically comprises a cannula body, one or two prongs extending from the body, and gases supply tubing. Although prongs for nasal cannula exist in the art, an aspect of at least one of the configurations disclosed herein includes the realisation that respiratory therapy or support may be improved by achieving increased patient pressure with a non-sealing nasal cannula. Currently it can be difficult to optimise dead space clearance and delivered pressure to the patient. Some options with existing interfaces may include providing undesirably high flows to patients to achieve a desired pressure effect. It may be desirable to achieve an increased patient pressure, such as peak end expiratory pressure (PEEP) with an open, or flow-controlled respiratory support system. It may also be desirable to provide a means to better control patient airway pressure with open, or flow-controlled systems.

An aspect of the present disclosure provides a prong for a patient interface, wherein the prong comprises:
a proximal end having an opening configured to deliver a gas into a naris;
a distal end configured to be in fluid communication with a gas source;
an inner surface defining a passage between the proximal end and the distal end;
an outer surface; and
at least one protrusion on the outer surface of the prong, wherein the at least one protrusion provides an increased resistance to a flow of exhaled gas along the outer surface of the prong.

In some configurations, at least one of:
the at least one protrusion causes a redirection of a flow of gas along the outer surface of the prong;
the at least one protrusion creates a turbulence in a flow of gas along the outer surface of the prong;
the at least one protrusion partially occludes a space between the outer surface of the prong and an inner surface of the naris;
the at least one protrusion creates a tortuous flow path for exhaled gas along the outer surface of the prong;
the at least one protrusion provides a continuous flow path for the exhaled gas along the outer surface of the prong;
the at least one protrusion increases the peak end expiratory pressure (PEEP) compared to the prong if no protrusions were present;
the prong comprises a plurality of the protrusions and the protrusions are arranged in at least one row
the prong comprises a plurality of protrusions in at least one row arranged substantially circumferentially around the outer surface of the prong;
the prong comprises a plurality of protrusions and at least one flow path for exhaled gas from the distal end to the proximal end is defined between the protrusions on the outer surface of the prong;
the prong comprises a plurality of protrusions arranged such that a narrowing of at least one flow path is created between two or more adjacent protrusions;
the prong comprises a series of narrowings of the at least one flow path between a plurality of pairs of adjacent protrusions;
the at least one protrusion provides a continuous flow path for the exhaled gas along the outer surface of the prong wherein the at least one flow path is non-linear;
the at least one protrusion comprises a wall that is arranged at least partially helically about the outer surface of the prong;
the prong comprises a plurality of the protrusions and the protrusions are arranged such that exhaled gas in the flow path is redirected a plurality of times;
the prong comprises a plurality of protrusions and a plurality of flow paths for exhaled gas are defined between the protrusions from the distal end to the proximal end;
a flow of exhaled gas within the naris has a bulk flow direction and the at least one protrusion is configured to redirect at least a portion of the flow back towards the bulk flow;
the at least one protrusion comprises a surface which is outwardly curved relative to the outer surface of the prong;
the at least one protrusion is hemispherical, substantially hemispherical, and/or has a rounded or curved profile;
the at least one protrusion has an outer surface that is outwardly curved relative to the outer surface of the prong;
the at least one protrusion is configured to redirect a flow of gas;
the at least one protrusion has a first surface facing the distal end of the prong, the first surface having side edges, and the at least one protrusion has side walls extending inwardly and towards the proximal end of the protrusion from the side edges;
the at least one protrusion is conical in shape or is at least partially conical in shape;
the at least one protrusion has a surface that is concave relative to the distal end of the prong;
the at least one protrusion has a first surface substantially facing towards the distal end of the prong, the first surface comprising an indentation;
the at least one protrusion is an elongate wall.
the at least one protrusion may extend in a longitudinal direction of the prong;
the at least one protrusion is an elongate wall extending substantially longitudinally between the distal end and proximal end of the prong;
the at least one protrusion extends substantially longitudinally partially between the distal end and the proximal end of the prong;
the prong comprises a plurality of protrusions and the protrusions are arranged in rows, optionally wherein the protrusions of each row are staggered relative to the protrusions of each adjacent row;
the at least one protrusion is an elongate wall extending from the outer surface of the prong and extending a partial distance around a circumference of the prong;
the at least one protrusion is a curved wall extending from the outer surface of the prong and extending a partial distance around a circumference of the prong;
the prong comprises a plurality of protrusions arranged in at least one row about a circumference of the prong; and/or
the prong comprises a plurality of protrusions arranged in a plurality of rows, where each row comprises at least one protrusion.

The following description may include reference to features of the prong and/or protrusions relative to a flow of gas. In those references it is understood that they relate to the prong when in use in a naris of a person. It is understood that any interaction or effect the prong and/or protrusion has with the flow of gas is due to the prong and/or protrusion being configured to provide that interaction or effect.

In some configurations, the prong comprises a plurality of rows that are aligned substantially perpendicularly about the outer surface of the prong. An edge of a protrusion in a first row may be aligned with an opposed edge of a protrusion in a second row. At least one protrusion in a first row may be aligned with at least one protrusion in a second row. At least one protrusion in a first row may be offset in alignment from at least one protrusion in a second row. At least one protrusion in a first row may overlap with at least one protrusion of a second row. Each protrusion in a first row may not overlap with each protrusion of a second row. According to any of these configurations, the first row and the second row may be adjacent rows.

In some configurations, when the prong is within the naris, the protrusion or protrusions on the outer surface of the prong is or are arranged to provide a continuous airflow path between the outer surface of the prong and the inner surface of the naris from the distal end to the proximal end of the prong.

In some configurations, when the prong is within a naris, a space is present between the outer surface of the prong and an inner surface of the naris. The protrusion(s) on the outer surface of the prong may contact the inner surface of the naris, when the prong is within a naris.

In some configurations, the prong comprises a plurality of protrusions.

In some configurations, a flow path for exhaled gas from the distal end to the proximal end is defined between the protrusions on the outer surface of the prong.

In some configurations, the protrusions may define a winding and/or tortuous path for an expelled flow of gas over the outer surface of the prong.

In some configurations, the protrusions may alter the velocity of exhaled gas over the outer surface of the prong. The protrusions may create localised variations in the velocity of portions of the flow of the exhaled gas over the outer surface of the prong. For example, each protrusion may create a localised reduction in the cross-sectional area between the outer surface of the prong and the inner surface of the naris compared to locations where no protrusion is present. Each protrusion may increase the outer circumference of the prong. Where the cross-sectional area is reduced there will be an increase in flow velocity to maintain the same average flow rate of the exhaled gas.

In some configurations, the protrusions may redirect at least a portion of the flow of exhaled gas. The at least portion of exhaled gas may be redirected back towards the oncoming flow of exhaled gas.

In some configurations, protrusions may cause an increased occlusion of the naris while maintaining a flow path for exhaled gas to escape the naris.

The prong may have a first outer circumference on the outer surface, of the prong, and a second outer circumference at an outer extent of the at least one protrusion. The second outer circumference may be larger than the first outer circumference. The larger second circumference may provide an increase in a ratio of cross-sectional prong area to cross-sectional naris area. The larger second circumference may provide increased occlusion at the outer extent of the protrusion when in use.

The prong may comprise a plurality of outer circumferences at different positions along the length of the prong. The size of the outer circumference at a given position may be dependent on any one or more of the following: the number of protrusions present at the respective position on the prong; the shape of the or each protrusion; the thickness of the material forming the prong body at that position; the overall shape of the prong; and any tapering of the prong.

In some configurations, the protrusions may be aligned with one another. The protrusions may be aligned in the longitudinal direction. At least one protrusion may be aligned with at least one other protrusion in the longitudinal direction. The protrusions may be aligned in the circumferential direction. At least one protrusion may be aligned with at least one other protrusion in the circumferential direction. The protrusions may be aligned at an angle oblique to the longitudinal direction. At least one protrusion may be aligned with at least one other protrusion at an angle oblique to the longitudinal direction.

In some configurations, the protrusions may be misaligned with respect to one another.

The protrusions may be positioned in a substantially random pattern over the outer surface of the prong.

The protrusions may be arranged in one or more rows. The row or rows may each extend circumferentially or substantially circumferentially about the outer surface of the prong. The row or rows may each extend about the outer surface of the prong substantially perpendicularly to the longitudinal direction. The row or rows may each extend about the outer surface of the prong. The row or rows may each extend about the outer surface of the prong at an angle to a circumference of the outer prong surface. The row or rows may each extend substantially along a longitudinal direction of the prong.

The protrusions may be offset relative to one another. The protrusions may be longitudinally offset on the outer surface relative to one another. The protrusions may be circumferentially offset relative to one another about the outer surface of the prong. A protrusion may be offset relative to an adjacent protrusion. A protrusion may be longitudinally offset relative to an adjacent protrusion. A protrusion may be circumferentially offset relative to an adjacent protrusion. A protrusion may be in line longitudinally with an adjacent protrusion. A protrusion may be in line circumferentially with an adjacent protrusion.

The protrusion or protrusions of a first row may be in line with the protrusion or protrusions of an adjacent row. The protrusion or protrusions of each row may be aligned with the protrusion or protrusions of each other row. The protrusion or protrusions of each row may be offset relative to the protrusion or protrusions of each adjacent row.

The protrusions may be arranged in rows about a circumference of the prong, with little or no overlap between protrusions of one row and the protrusions of an adjacent row. The protrusions may be arranged in rows about a circumference of the prong with little or no overlap between protrusions of one row and the protrusions of each other row.

A first row of protrusions may contain a different number of protrusions to a second row. A row may contain the same number of protrusions as at least one other row. Alternate rows of protrusions may have the same number of protrusions as one another. Each row of protrusions may have a different number of protrusions to each adjacent row of protrusions. A row of protrusions may contain the same number of protrusions as at least one other row of protrusions. Each row of protrusions may comprise the same number of protrusions as each other row of protrusions.

At least one row may include at least one protrusion, at least two protrusions, at least three protrusions, at least four protrusions, about three protrusions, about four protrusions, about five protrusions, about six protrusions, about seven protrusions, about eight protrusions, about nine protrusions, about ten protrusions, at least ten protrusions, about eleven protrusions, about twelve protrusions, about fifteen protrusions, about sixteen protrusions, about twenty protrusions, at least twenty protrusions, about twenty five protrusions.

At least one protrusion may have a curved surface relative to the outer surface of the prong. At least one protrusion may have an outer surface that is outwardly curved relative to the outer surface of the prong. At least one protrusion may have a rounded surface. At least one protrusion may be a hemispherical protrusion, substantially hemispherical protrusion, or a partially hemispherical protrusion. Each protrusion may be a hemispherical, substantially hemispherical, or partially hemispherical protrusion. Each protrusion may have a curved surface relative to the outer surface of the prong. Each protrusion may have an outer surface that is outwardly curved relative to the outer surface of the prong. Each protrusion may have a rounded surface.

The or each or at least one protrusion may have a proximal end and a distal end.

At least one protrusion may be configured to redirect at least a portion of a flow of exhaled gas back on itself. The at least one protrusion may have a first surface facing the distal end of the prong. The first surface may have side edges. The at least one protrusion may have side walls extending from the side edges. The side walls may extend inwardly and towards the proximal end of the protrusion from the side edges. The first surface of the protrusion may be curved or may comprise a curved face. The curve of the first surface or curve of the curved face may be concave relative to the distal end of the prong. The protrusions may be arranged in rows. Protrusions of one row may be staggered relative to protrusions of an adjacent row.

The flow of exhaled gas may travel generally in a substantially longitudinal direction between the outer surface of the prong and the inner surface of the naris towards an opening of the naris. An average flow direction of the exhaled gas may be towards the opening of the naris. The average flow direction may be generally longitudinally from the distal end to the proximal end of the prong when the prong is inserted in the naris. The flow of exhaled gas may have an average flow velocity and/or an average flow rate.

At least one protrusion may be shaped or configured to redirect at least a portion of a flow of the exhaled gas. The at least portion of the flow of exhaled gas may be redirected by the at least one protrusion back towards the flow of gas flowing in the average flow direction, The protrusion(s) may redirect at least a portion of the flow of gas in a direction opposed to the average flow direction.

The protrusion may comprise a first wall or first surface on the outer surface of the prong. The first wall of the protrusion may also be referred to as a first surface herein. The first wall may be substantially perpendicular to the longitudinal direction between the proximal end and the distal end of the prong. The first wall of the protrusion may extend substantially in the circumferential direction of the prong. The first wall may comprise an angled or curved surface. The first wall of the protrusion may be outwardly curved between its first and second ends relative to the distal end of the prong.

The first wall may be opposed to the average flow direction. The first wall of the protrusion may create a localised reduction in the cross-sectional area between the outer surface of the prong and the inner surface of the naris. In other words, the first wall of the protrusion may create a localised increase in occlusion of the naris. A portion of the flow of exhaled gas may interact with the first wall of the protrusion. The first wall of the protrusion may redirect at least a portion of the flow of exhaled gas. The first wall of the protrusion may cause a turbulence in the flow of exhaled gas. The protrusion may cause localised changes in the velocity of the flow of exhaled gas.

The protrusion may comprise second and third walls. The second and third walls may extend along the surface of the prong from opposed first and second ends of the first wall. The second and/or third walls may extend at an angle to the longitudinal direction towards the proximal end. The angle to the longitudinal direction may be acute. The second and third walls may meet one another at a location towards the proximal end of the prong relative to the first wall. The second and third walls may meet at a point or corner. The point where the second and third walls meet may be angled. The point where the second and third walls meet one another may be curved. The second and third walls may also be termed as side walls herein. The protrusion comprising the first, second and third walls may be shaped similar to a triangle.

An outer surface of the protrusion may be provided between outermost edges of the first, second and third walls. The outer surface of the protrusion may be substantially flat or planar. The outer surface of the protrusion may be curved or rounded. The edges where the outer surface of the protrusions meets the first, second and third walls may be angled or may be curved.

At least one protrusion may have a conical shape. The at least one protrusion may be a conical protrusion. The at least one protrusion may have a semi-conical shape. The conical protrusion may be shaped similar to a cone that is bisected along its length. A base of the conical protrusion may be positioned nearest to the distal end of the prong compared to the rest of the conical protrusion. The base may extend substantially perpendicularly from the outer surface of the prong. The base may have a substantially semi-circular shape. A tapered wall of the conical protrusion may extend from an edge of the base towards a point on the outer surface of the prong towards the proximal end. The tapered wall may meet the base of the protrusion with a curved transition.

The or each protrusion may reduce the space available between the prong and the inner surface of the naris for the exhaled gas to flow towards an opening of the nose, which may result in a higher pressure within the nose and/or patient airway. The protrusions may be positioned in a staggered pattern which may create a winding exhaled gas flow path. Staggering the protrusions relative to one another may also increase the interactions between the flow of exhaled gas flowing generally in the average flow direction with the protrusions. The staggered pattern of protrusions may decrease the dynamic pressure of the exhaled gas and increase the static pressure. The staggered pattern of protrusions may increase the dynamic pressure of the exhaled gas and decrease the static pressure.

The at least one protrusion may have a first surface substantially facing towards the distal end of the prong. The first surface may be opposed to the average flow direction of the flow of exhaled gas. The first surface of the protrusion may be curved. The first surface of the protrusion may comprise a concave face relative to the average flow direction of exhaled gas. The concave face may be on the first surface of the protrusion. At least a portion of the exhaled gas may be redirected by the concave face. The concave face may extend substantially outwardly from the outer surface of the prong.

A trailing surface may extend from an outer edge of the first surface towards the proximal end of the prong. The trailing surface may extend inwardly from the outer edge of the first surface towards the outer surface of the prong in the proximal direction. The trailing surface may be curved from the outer edge of the first surface towards the outer surface of the prong. The trailing surface may be flat or planar. Each side of the protrusion may comprise a side wall extending substantially perpendicularly from the outer surface of the prong. The side wall may extend between the outer surface of the prong, the first surface and the trailing surface.

The concave face may redirect a portion of the exhaled gas back towards exhaled gas flowing in the average flow direction. Redirection of the flow of exhaled gas may increase turbulence. Eddies may be induced in the flow of exhaled gas. The eddies may modify the velocity of a portion of the exhaled gas and/or may increase peak end expiratory pressure (PEEP) relative to a prong with no protrusions present on the outer surface. Where a portion of the exhaled gas is caused to reduce in velocity by the protrusion(s) another portion of the exhaled has may increase in velocity such that the average flow velocity of the exhaled gas is maintained.

Reference is made herein to increased pressure or increased PEEP by protrusions on the outer surface of a prong. What is meant by this is an increase in the pressure/PEEP in comparison to a prong of the same shape and dimensions (circumference, inner diameter, outer diameter, wall thickness, height, length) but having no protrusions on its outer surface, for a given flow rate.

The trailing surface of the protrusion may aid in pulling gas towards the outer surface of the prong. This may cause at least a portion of the exhaled gas to flow in close proximity to the outer surface of the prong.

The at least one protrusion may have a shark fin shape in longitudinal cross-section. The at least one protrusion may have a shark fin shaped cross-section that is substantially the same across the width of the protrusion.

The prong may comprise a plurality of elongate protrusions aligned substantially in a longitudinal direction of the prong. One or more elongate protrusions may be an elongate cuboid or approximate an elongate rectangular prism. One or more elongate protrusions may have curved or rounded edges. Each elongate protrusion may have the same general shape as each other elongate protrusion. The protrusions may extend between the distal end and proximal end of the outer surface of the prong. The protrusions may be aligned in a single row about the circumference of the prong. At least one wall of the protrusion may extend substantially perpendicularly from the outer surface of the prong. The protrusions may extend substantially perpendicularly from the outer surface of the prong.

The protrusion may reduce the space between the outer surface of the prong and the inner surface of the naris, compared to a prong of equivalent dimensions with no protrusions. The reduced space in the naris for exhaled gas to flow may result in a higher pressure within the naris and/or airway of the patient. Each protrusion may cause a localised narrowing of available space within the naris. This may lead to one or more regions of turbulence in the flow of exhaled gas within the naris. The localised narrowing may cause an irrecoverable loss of pressure due to the regions of turbulence and/or eddies that may be induced in the flow.

At least one protrusion may be or may approximate a rectangular prism shape. At least one protrusion may have at least one curved or rounded edge. At least one protrusion may be a non-rectangular prism. The at least one protrusion may be a substantially square sided prism. The at least one protrusion may be a block or cuboid shape. At least one side wall of the at least one protrusion may extend substantially perpendicularly from the outer surface of the prong. At least one side wall may extend outwardly at an angle to the outer surface of the prong. An outer end surface of the at least one protrusion may extend between the edges of the side wall or side walls distanced from the outer surface of the prong.

At least one protrusion may be elongate in the circumferential direction. At least one protrusion may comprise an elongate wall. The protrusion may extend substantially circumferentially about the prong. The at least one protrusion may extend substantially perpendicularly outwards form the outer surface of the prong. The protrusion may have a wall surface extending outwardly from the outer surface of the prong and being elongate in the circumferential direction. The wall surface may oppose the flow of exhaled gas travelling in the average flow direction.

An increase to the width/length of a protrusion in the circumferential direction may increase PEEP. The wider the protrusion is in the circumferential direction, the closer the protrusion may be to becoming a ring. A complete ring-like structure extending about the entire circumference may generate high PEEP relative to protrusion that does not extend about the entire circumference.

However, it may pose a risk of completely sealing a naris, which may detract from respiratory therapies, for example, high flow therapy t which utilises an unsealed system. Therefore, utilising a wide protrusion in the circumferential direction, but maintaining at least one gap in the protrusion circumferentially is desirable to prevent the naris from being fully occluded. This may ensure a flow path is maintained for exhaled gas.

A tortuous path may be provided when circumferentially elongate protrusions are in the staggered arrangement. This may result in higher PEEP being generated as exhaled gas undergoes sudden expansion and contraction compared to a comparable prong where no protrusions are present. The exhaled gas may be caused to change directions in between the staggered protrusions. Providing a path of high resistance to the exhaled gas may increase PEEP provided to the patient.

In some configurations, the at least one protrusion includes a curved wall extending from the outer surface of the prong. In some configurations, at least one protrusion comprises an elongate curved wall which extends substantially circumferentially about the prong. The at least one protrusion may comprise an elongate structure that is curved about its centre. Each end of the elongate structure may point at least partially towards the distal end of the prong. The elongate curved wall of the protrusion may be configured to redirect a portion of the flow of exhaled gas.

In some configurations, the prong comprises a single protrusion.

In some configurations, at least one protrusion may be a helical protrusion. In some configurations, each one of a plurality of protrusions is a helical protrusion. The or each helical protrusion may be helically arranged between the distal end and the proximal end about the outer surface of the prong. The or each protrusion may extend in both the longitudinal and circumferential directions.

The or each protrusion may extend helically about the prong by any numbers of revolutions, for example: from a tenth of a revolution to a half a revolution; from a tenth of a revolution to a whole revolution; from one to fifty revolutions; one to twenty revolutions; one to ten revolutions; one to five revolutions; two to twenty revolutions; two to ten revolutions; two to six revolutions; less than twenty revolutions; less that ten revolutions; at least one revolution; at least two revolutions; at least five revolutions; or any number of revolutions less than, between, or above any of these numbers of revolutions.

Utilising at least one helical protrusion may provide the exhaled gas with a tortuous path by creating a winding path of exit. This may increase the length of the exit path of the exhaled gas from the naris. Increasing the number of helical protrusions may result in increased PEEP compared to a smaller number of helical protrusions. Increasing the number of revolutions about the prong by the or each protrusion may result in increased PEEP compared to fewer revolutions.

In some configurations, the at least one protrusion causes a redirection of the flow of exhaled gas.

In some configurations, the at least one protrusion promotes turbulence in the flow of exhaled gas.

In some configurations, the at least one protrusion partially occludes a space between the outer surface of the prong and an inner surface of the naris without sealing the naris. The protrusion may maintain a flow path for exhaled in the naris between the distal end and the proximal end of the prong.

In some configurations, the at least one protrusion creates a tortuous flow path for the exhaled gas along the outer surface of the prong.

In some configurations, the at least one protrusion ensures a continuous flow path for the exhaled gas along the outer surface of the prong.

In some configurations, the prong having at least one protrusion increases the peak end expiratory pressure (PEEP) in comparison to a comparable prong with no protrusions. A ‘comparable prong’ in this context is a prong that is of substantially the same size dimensions, including circumference at the prong outer surface, internal diameter, outer diameter, prong length and wall thickness. The difference is that one prong has protrusions extending from its outer surface and the comparable prong has no protrusions.

In some configurations, the prong comprises a plurality of protrusions and a flow path for the exhaled gas in the naris between the distal end to the proximal end of the prong is provided between the protrusions on the outer surface of the prong. The protrusions may be arranged such that a narrowing in a section of the flow path is created between two or more adjacent protrusions compared to at least one other section of the flow path on the outer surface of the prong.

In some configurations, the prong comprises a plurality of protrusions and a flow path for the exhaled gas in the naris from the distal end to the proximal end is provided between the protrusions on the outer surface of the prong. The protrusions may be arranged such that exhaled gas in the flow path is redirected a plurality of times. The protrusions may interact with the flow of exhaled gas such that the flow of gas is caused to be redirected a plurality of times between the distal end and the proximal end of the prong.

In some configurations, the protrusions are arranged such that a flow path of the exhaled gas between the distal and the proximal end of the prong is non-linear.

In some configurations, the prong comprises a plurality of protrusions and a plurality of flow paths for the exhaled gas are provided by the protrusions between the distal end and the proximal end of the prong.

In some configurations, the protrusion or protrusions act to redirect a flow of exhaled gas along the outer surface of the prong.

In some configurations, the protrusion or protrusions may act to reduce a velocity of a flow of exhaled gas along the outer surface of the prong. In some configurations, the protrusion or protrusions may lead to localised increases and decreases to the velocity of at least portions of a flow of exhaled gas in the naris.

In some configurations, the protrusion or protrusions act to reduce a dynamic pressure and to increase a static pressure of exhaled gas.

In some configurations, the protrusion or protrusions act to increase PEEP provided to a patient in whose nose the prong is inserted, compared to a prong with no protrusions.

In some configurations, the protrusion or protrusions may redirect a flow of exhaled gas. Redirection of the flow of exhaled gas may create mixing of the exhaled gas. Redirection of the flow of exhaled gas may increase turbulence in the flow. The protrusion or protrusions may act to induce at least one eddy in the flow of exhaled gas. The eddy or eddies may increase resistance to flow of the exhaled gas and/or may increase PEEP in comparison to a comparable prong without protrusions.

In some configurations, the protrusion or protrusions on the prong provide an occlusion percentage of the naris within the range of 40% to 90%, 45% to 80%, 50% to 70%, less than 100%, less than 90%, or less than 80%.

In some configurations a prong having at least one protrusion according to the present disclosure may be formed from a soft, flexible and/or elastomeric material, for example, silicone, thermoplastic elastomers, or other polymers known in the art. The prong and protrusion(s) may be formed from any type of material that provides a soft interfacing component for comfortably delivering the flow of gases to a person through the prongs, whilst maintaining structure of the protrusions.

A patient interface comprising at least one prong and protrusion may be formed at least partially from a soft, flexible and/or elastomeric material, for example, silicone, thermoplastic elastomers, or other polymers known in the art. The patient interface may be formed at least partly from the same material as the prong and/or protrusion(s).

The prong may be about 10 mm in length. The prong may be less than 20 mm in length. The thickness of prong may be about 5-10% of the length of the prong. The thickness of the prong may be about 0.5 mm to 1 mm. The thickness of the prong referred to may be the thickness of a wall of the prong between the outer surface and inner surface of the prong.

An aspect of the present disclosure provides a patient interface comprising a prong as described in any one or more of the aspects, configurations and/or examples provided herein. The patient interface may be a nasal interface, such as a nasal cannula.

Another aspect of the present disclosure provides a patient interface comprising a pair of prongs as described in any one or more of the aspects, configurations and/or examples provided herein.

The patient interface may comprise a manifold comprising at least one gases inlet for delivery of respiratory gases to the manifold. The prongs of the patient interface may share a common manifold.

In some configurations, at least one gases inlet is at a side of the manifold.

The patient interface may be configured to receive a breathable gas from a therapy support system via at least one tube. The at least one tube may supply the breathable gas to the manifold of the patient interface.

The patient interface may comprise at least one wing or arm. The wing(s) or arm(s) may comprise or be attachable to at least one dermal patch configured to removably attach the patient interface to a patient. The at least one wing or arm of the patient interface may comprise an opening to receive a breathable gas via the tube. The patient interface may comprise a pair of the wings or the arms. The patient interface may receive the breathable gas via an opening in each wing or arm each connectable to a respective tube.

The patient interface may be provided with a structure to direct the flow of breathable gas from the or each tube to the prongs. The structure may comprise the manifold. The structure within the patient interface may fluidly connect the pair of prongs. The structure within the patient interface may be configured to provide a flow of gas from a separate tube to each prong.

The patient interface according to any of the above examples may be a nasal cannula.

In accordance with a further aspect, a respiratory system for providing a breathable gas to patient is disclosed, the respiratory therapy system comprising:
a gases source for respiratory gases;
a breathing tube to receive the respiratory gases; and
a nasal interface comprising a prong as described in any one or more of the aspects, configurations and/or examples herein.

In some configurations, the respiratory therapy system comprises a nasal interface comprising a pair of prongs as described in any one or more of the aspects configurations and/or examples herein.

The gases source for respiratory gases may be configured to provide flow controlled respiratory gases.

In some configurations, the respiratory therapy system comprises a respiratory conduit to receive the respiratory gases from the breathing tube, wherein the respiratory conduit is in fluid communication with the breathing tube and the gases inlet of the nasal interface.

In some configurations, the respiratory therapy system further comprises a humidifier configured to humidify said respiratory therapy gases prior to their delivery to the nasal interface.

In some configurations, the breathing tube is a heated breathing tube.

In some configurations, the respiratory therapy system delivers high flow therapy to the patient.

There is also provided herein a method of providing respiratory support to a patient, the method comprising: providing a respiratory therapy system comprising: a gases source for respiratory gases; a breathing tube to receive the respiratory gases; and a patient interface having a gases inlet in fluid communication with the breathing tube to deliver the respiratory gases to a patient, the patient interface comprising at least one prong having at least one protrusion on an outer surface of the prong; locating the at least one prong in a naris of the patient in a non-sealing manner; operating the respiratory therapy system to provide a flow of gases to the patient interface; and delivering a flow of gases from the respiratory therapy system through the at least one prong at a naris of the patient, wherein the at least one protrusion provides an increased resistance to a flow of exhaled gas from the naris of the patient.

The method may comprise a patient interface as disclosed herein. The patient interface may be a nasal interface, for example a nasal cannula.

The method may comprise a respiratory therapy system as disclosed herein.

An aspect of the present disclosure provides a prong for a nasal cannula, wherein the prong comprises: an outer surface; a proximal end; a distal end; an inner surface defining a passage between the proximal end and the distal end of the prong; and at least one protrusion on the outer surface of the prong, wherein, in use, the prong is insertable into a naris and allows a flow of exhaled gas along the outer surface of the prong, and wherein at least one of the following:
In some configurations, the at least one protrusion causes a redirection of the flow of exhaled gas;
In some configurations, the at least one protrusion creates a turbulence in the flow of exhaled gas;
In some configurations, the at least one protrusion partially occludes a space between the outer surface of the prong and an inner surface of the naris without sealing the naris;
In some configurations, the at least one protrusion creates a tortuous flow path for the exhaled gas along the outer surface of the prong;
In some configurations, the at least one protrusion provides a continuous flow path for the exhaled gas along the outer surface of the prong;
In some configurations, the at least one protrusion increases the peak end expiratory pressure (PEEP);
In some configurations, the prong has a plurality of the protrusions and the protrusions are arranged in a plurality of rows substantially circumferentially around the outer surface of the prong;
In some configurations, the prong has a plurality of the protrusions and a flow path for the exhaled gas from the distal end to the proximal end is provided between the protrusions on the outer surface of the prong, the protrusions being arranged such that a narrowing of the flow path is created between two or more adjacent protrusions compared to at least one other section of the flow path on the outer surface of the prong;
In some configurations, the at least one protrusion comprises a wall that is arranged at least partially helically about the outer surface of the prong;
In some configurations, the prong has a plurality of the protrusions and a flow path for the exhaled gas from the distal end to the proximal end is provided between the protrusions on the outer surface of the prong, the protrusions being arranged such that exhaled gas in the flow path is redirected a plurality of times;
In some configurations, the prong has a plurality of the protrusions and a flow path for the exhaled gas from the distal end to the proximal end is provided between the protrusions on the outer surface of the prong, the protrusions being arranged such that the flow path is non-linear;
In some configurations, the prong has a plurality of the protrusions and a plurality of flow paths for the exhaled gas are provided by the protrusions between the distal end and the proximal end;
In some configurations, the flow of exhaled gas has an average flow direction, and wherein the at least one protrusion is shaped to redirect at least a portion of the flow of exhaled gas back towards exhaled gas travelling in the average flow direction;
In some configurations, the prong has a plurality of the protrusions and the protrusions have a curved or rounded outer profile;
In some configurations, the prong has a plurality of the protrusions and the protrusions are hemispherical or partially hemispherical;
In some configurations, the at least one protrusion is substantially or partially conical in shape;
In some configurations, the at least one protrusion has a concave face relative to the flow of exhaled gas such that at least a portion of the exhaled gas is redirected by the concave face;
In some configurations, the prong has a plurality of the protrusions, each protrusion being elongate and extending substantially longitudinally at least partially between the distal end and the proximal end of the prong;
In some configurations, the prong has a plurality of the protrusions and the protrusions are arranged circumferentially offset from one another in a longitudinal direction of the prong;
In some configurations, the at least one protrusion is elongate and extends substantially circumferentially a partial distance of a circumference of the prong;
In some configurations, the at least one protrusion has a curved wall which is configured to oppose a flow of gas flowing in an average flow direction;
In some configurations, the prong has a plurality of the protrusions and the protrusions are arranged in rows about a circumference of the prong, wherein an edge of a protrusion in a first row is aligned with an opposed edge of a protrusion in a second row;
In some configurations, the prong has a plurality of the protrusions and the protrusions are arranged in rows about a circumference of the prong, wherein a protrusion in a first row is aligned with a protrusion in a second row;
In some configurations, the prong has a plurality of the protrusions and the protrusions are arranged in rows about a circumference of the prong, wherein an protrusion in a first row is staggered relative to at least one protrusion in a second row; and/or
In some configurations, the prong has a plurality of the protrusions and the protrusions are arranged in rows about a circumference of the prong, wherein each protrusion of a first row overlaps with a protrusion of an adjacent second row.

An aspect of the present disclosure provides a nasal interface for delivery of respiratory gases, the nasal interface comprising:
a first prong having a first outer surface, and
a second prong having a second outer surface,
wherein the first prong has at least one protrusion on the first outer surface.

In some configurations, the first prong comprises a plurality of protrusions arranged on the first outer surface.

A nasal interface for delivery of respiratory gases, the nasal interface comprising:
at least one non-sealing nasal prong having a distal end and a proximal end;
the at least one non-sealing nasal prong comprising a plurality of protrusions on an outer surface of the non-sealing nasal prong.
wherein the plurality of protrusions comprises a first row of circumferentially spaced apart protrusions and one or more further rows of circumferentially spaced apart protrusions.

In some configurations, the protrusions of the first row are staggered/offset relative to the protrusions of at least one adjacent row.

In some configurations, each protrusions of the first row may overlap with a protrusion of at least one adjacent row circumferentially

In some configurations, a side edge of a protrusion of the first row is aligned with an edge of a protrusion of at least one adjacent row.

In some configurations, the protrusions of the first row are aligned relative to the protrusions of at least one adjacent row.

In some configurations, the second outer surface has a first outer circumference, and an outer extent of the prong at the at least one protrusion has a second outer circumference, wherein the second outer circumference is greater than the first outer circumference.

In some configurations, at least one protrusion is elongate in the circumferential direction.

In some configurations, at least one protrusion is elongate in the longitudinal direction.

In some configurations, the at least one protrusion is elongate and aligned obliquely to the circumference of the first prong.

In some configurations, the at least one protrusion has a substantially rectangular cross-sectional shape.

In some configurations, at least one protrusion comprises an outer wall spaced from the first outer surface by a height of the protrusion.

In some configurations, the at least one protrusion comprises:
a leading face extending from the first outer surface and facing substantially towards the distal end of the at least one non-sealing nasal prong;
a trailing face extending from the first outer surface and facing substantially towards the proximal end of the at least one non-sealing nasal prong.

In some configurations, the at least one protrusion comprises side walls extending from the first outer surface and between the leading face and the trailing face.

In some configurations, the outer wall extends between the leading surface, the trailing surface and the side walls.

In some configurations, the side walls are substantially parallel to a longitudinal direction of the first prong.

In some configurations, the side walls extend away from one another between distal and proximal ends of the at least one non-sealing nasal prong.

In some configurations, the side walls extend towards one another between distal and proximal ends of the at least one non-sealing nasal prong.

In some configurations, the side walls extend towards one another outwardly from the first outer surface.

In some configurations, one or more of the leading face, trailing face, side walls and outer wall are curved, planar, convex or concave.

In some configurations, the leading face is at least one of curved or concave.

In some configurations, the at least one protrusions is polygonal.

In some configurations, the at least one protrusion is rounded.

In some configurations, each row of protrusions comprises the same number of protrusion.

In some configurations, each row of protrusions has at least 2, at least 3, at least 5, at least 8, at least 10, at least 12, at least 15 or at least 20 protrusions in it.

In some configurations, the first prong comprises at least 2, at least 3, at least 4, at least 5, at least 8, at least 10, at least 12, at least 15 or at least 20 rows of protrusions.

In some configurations, a spacing between protrusions in each row is constant.

In some configurations, a spacing between protrusions in each row varies.

In some configurations, a spacing between rows of protrusions is constant.

In some configurations, a spacing between rows of protrusions in varies.

In some configurations, the protrusions are substantially rigid and/or only partially flexible and/or not flexible.

In some configurations, the protrusions are arranged on the first outer surface to provide at least one flow path for a gas between the distal and proximal ends of the first prong.

In some configurations, the protrusion or protrusions on the prong provide an occlusion percentage of a naris within the range of 40% to 90%, 45% to 80%, 50% to 70%, less than 100%, less than 90%, or less than 80%.

In some configurations, the second prong comprises at least one protrusion on the first outer surface.

In some configurations, the second prong comprises no protrusion on the first outer surface and/or the first outer surface is smooth.

An aspect of the present disclosure provides a nasal interface for delivery of respiratory gases, the nasal interface comprising:
at least one non-sealing nasal prong
the at least one non-sealing nasal prong comprising a plurality of protrusions on an outer surface of the non-sealing nasal prong;
wherein the plurality of protrusions are arranged in a staggered pattern on the outer surface.

In some configurations, the plurality of protrusions overlap in the staggered pattern.

An aspect of the present disclosure provides a nasal interface comprising:
a pair of asymmetrical nasal prongs, each configured for delivery of respiratory gases to a naris of a patient, comprising
a first nasal prong; and
a second nasal prong;
wherein the first nasal prong has at least one protrusion on an outer surface of the first nasal prong; and
the second nasal prong has a substantially smooth outer surface and/or comprises no protrusions on its outer surface.

In some configurations, the first nasal prong and second nasal prong have the same internal cross-sectional area.

In some configurations, the at least one protrusion on the outer surface of the first nasal prong provides an increased external cross-sectional area compared to the outer surface of the first nasal prong where no protrusion is present.

In some configurations, each row of protrusions comprises the same number of protrusion.

In some configurations, a spacing between protrusions in each row is constant.

In some configurations, a spacing between protrusions in each row varies.

In some configurations, a spacing between rows of protrusions is constant.

In some configurations, a spacing between rows of protrusions varies.

In some configurations, the protrusions are arranged on the first outer surface to provide at least one flow path for a gas between the distal and proximal ends of the first prong.

An aspect of the present disclosure provides a nasal interface comprising:
a pair of asymmetrical nasal prongs, each configured for delivery of respiratory gases to a naris of a patient, comprising:
a first nasal prong; and
a second nasal prong;
wherein the first nasal prong has at least one protrusion on an outer surface of the first nasal prong, the at least one protrusion being in a first protrusion configuration; and
the second nasal prong has at least one protrusion on an outer surface of the second nasal prong, the at least one protrusion being in a second protrusion configuration,
wherein the first protrusion configuration is different to the second protrusion configuration.

In some configurations, the first protrusion configuration provides a first prong cross-sectional area that is greater than a second prong cross-sectional area

In some configurations, the first protrusion configuration comprises a first protrusion shape and wherein the second protrusion configuration comprises a second protrusion shape different to the first protrusion shape.

In some configurations, the first nasal prong provides a larger naris occlusion than the second nasal prong.

In some configurations, at least the second nasal prong is non-sealing to a naris.

In some configurations, the first protrusion configuration includes a first number of protrusions and the second protrusion configuration includes a second number of protrusions less than the first number.

In some configurations, the first protrusion configuration comprises a first number of circumferential rows of protrusions and the second protrusion configuration comprises a second number of circumferential rows of protrusions less than the first number.

In some configurations, the first protrusion configuration comprises a first number of circumferential rows of protrusions and the second protrusion configuration comprises a second number of circumferential rows of protrusions less than the first number.

In some configurations, the first protrusion configuration comprises a plurality of first protrusions arranged in first rows, wherein the first protrusions are offset from one another in adjacent first rows, and the second protrusion configuration comprises a plurality of second protrusions arranged in second rows, wherein the second protrusions are aligned with protrusions of each adjacent second row.

In some configurations, the first protrusion configuration comprises a first protrusion height and the second protrusion configuration comprises a second protrusion height smaller than the first protrusion height.

In some configurations, the first protrusion configuration comprises a first protrusion circumferential width and the second protrusion configuration comprises a second protrusion circumferential width smaller than the first protrusion circumferential width.

In some configurations, the first protrusion configuration comprises a first protrusion longitudinal length and the second protrusion configuration comprises a second protrusion longitudinal length smaller than the first protrusion longitudinal length.

Any feature of the above described aspects, configurations or examples may be combined with a feature or features of any other aspect(s), configuration or example(s) as described herein. The above statements provide defining features that may be combined together and/or with any one or more of the features of the configurations, examples, and/or aspects as provided herein.

### DEFINITIONS

### Circumference

In the present disclosure the term circumference, circumferential or derivative terms are used in respect to a prong. These terms are used to refer to a distance, direction and/or line around the prong. The use of these terms in respect of the prong does not make any implication herein to the outward or cross-sectional shape for the prong. For example, the prong may have an outward or cross-sectional shape that is substantially circular, substantially elliptical, substantially oval, substantially square, substantially rectangular, substantially triangular or any other possible shape that may have one or more curved and/or one or more straight edges.

### BRIEF DESCRIPTION OF DRAWINGS

Examples of the disclosure will now be described with reference to the accompanying drawings. It is to be understood that the examples are given by way of illustration only and the disclosure is not limited by this illustration. In the drawings:

FIGS. 1, 2 and 3 show cross-sectional representations of partial occlusion of a representation of a naris provided by a prong according to examples of the present disclosure;

FIG. 4 shows a schematic representation of an example respiratory support system;

FIG. 5 shows a front perspective view of a body of an example nasal cannula including facial pads;

FIG. 6 shows a front perspective view of an example nasal cannula affixed to a neonatal patient;

FIG. 7 shows an exploded view of the nasal cannula of FIG. 6;

FIG. 8 shows a side view of an example of a prong with hemispherical protrusions;

FIG. 9 shows a representation of fluid flow over the prong of FIG. 6;

FIG. 10 shows a side view of an example of a prong with protrusions configured to redirect portions of the flow of exhaled gas;

FIG. 11 shows a representation of fluid flow over the prong of FIG. 10;

FIG. 12 is an enlarged view of section B of FIG. 11;

FIG. 13 shows a side view of an example of a prong with cone shaped protrusions;

FIG. 14 shows a representation of fluid flow over the prong of FIG. 13;

FIG. 15 shows a perspective view of an example of a prong with a further configuration of protrusions;

FIGS. 16, 17, 18 show perspective views of examples of a prong with elongate longitudinally aligned protrusions;

FIG. 19 shows a perspective view of an example of a prong with cuboid protrusions;

FIG. 20 shows a perspective view of another example of a prong with cuboid protrusions;

FIG. 21 shows a side view of an example of a prong with wall protrusions and FIG. 22 shows an end view of the same prong with wall protrusions;

FIG. 23 shows a side view of another example of a prong with wall protrusions and FIG. 24 shows an end view of the same prong with wall protrusions;

FIG. 25 shows a side view of another example of a prong with wall protrusions and FIG. 26 shows an end view of the same prong with wall protrusions;

FIG. 27 shows a schematic end view of a prong with wall protrusions that has no overlap between protrusions in adjacent rows;

FIG. 28 shows a schematic end view of a prong with wall protrusions that has edges of protrusions in adjacent rows that are aligned;

FIG. 29 shows a schematic end view of a prong with wall protrusions with an overlap between protrusions in adjacent rows;

FIG. 30 shows a side view of an example of a prong with curved wall protrusions;

FIG. 31 shows a side view of an example of a prong with wall protrusions that have a concave surface facing an average flow direction of exhaled gas;

FIG. 32 shows a perspective view of an example of a prong with a plurality of helical protrusions;

FIGS. 33, 34, 35, 36 show perspective views of examples of prongs with a single helical protrusion;

FIGS. 37 and 38 are perspective views of an example of a patient interface in the form of a nasal cannula comprising a pair of prongs having protrusions;

FIG. 39 is a perspective view of another example of a patient interface comprising a pair of prongs having protrusions; and

FIG. 40 shows prongs of a patient interface where prong geometry differs between each prong.

### DETAILED DESCRIPTION

Patient interfaces can be used for delivering breathing gases to airways of a patient. The patient interfaces may comprise nasal interfaces that can be used to deliver a flow of gases to a patient. Nasal delivery elements, such as nasal prongs, may be inserted into one or both nares of a patient to deliver the required therapy. The nasal delivery elements may be desired to be non-sealing at one or both nares to deliver the therapy.

Disclosed is a system to deliver respiratory gases to a patient through a nasal interface.

Respiratory gases may include gas mixture compositions including supplemental oxygen and/or administration of therapeutic medicaments.

The system may utilise a non-sealing nasal interface to deliver a flow of gases to the patient. The system may be a non-invasive therapy.

The therapy may be flow-rate based and/or may be delivered by setting a flow rate. A predictable pressure may be achieved at a given flow rate.

As an example, the system may be configured to deliver high flow therapy. High flow therapy as discussed herein is intended to be given its typical ordinary meaning, which generally refers to a respiratory system delivering a targeted flow of respiratory gases via an intentionally unsealed patient interface, with flow rates generally intended to meet or exceed inspiratory flow of a patient.

High flow therapy is a flow-based therapy or form of respiratory support that may include a flow source to provide a flow of gases comprising air and/or oxygen and a patient interface to deliver breathable gas to the patient. A humidifier may be used to heat and humidify the flow of gases.

Typical flow rates for adults may range from, but are not limited to, about 15 litres per minute to about 60 litres per minute or greater. For example, for an adult patient ‘high flow therapy’ may refer to the delivery of gases to a patient at a flow rate of greater than or equal to about 10 litres per minute (10 litres per minute (LPM)), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM.

Typical flow rates for paediatric users, such as neonates, infants and children, often range from, but are not limited to, about 1 litre per minute per kilogram of patient weight to about 3 litres per minute per kilogram of patient weight or greater. For example, for paediatric patients, ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with a neonatal, infant, child or adult patient may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing the work of breathing. High flow therapy may generate a flushing or clearance effect in the nasopharynx. Fresh air replaces the carbon dioxide rich air, reducing the dead space of the airway.

Flow-based or flow-controlled respiratory therapies such as high flow therapy can also optionally include gas mixture compositions including supplemental oxygen and/or administration of therapeutic medicaments.

Patient airway pressure may be used to provide breathing assistance. Pressure-based therapies, such as continuous positive airway pressure therapy (CPAP), are the ‘gold standard’ for treating respiratory failure. These therapies use a patient interface that forms a seal with the patient airway, i.e. a ‘sealing’ interface. Sealing interfaces may be associated with difficulties such as air leaks, patient discomfort and/or skin damage. Mitigating such problems often depends on proper fitting of the sealing interface, which may be difficult.

The scope of the present disclosure is not limited to high flow applications and may be used with any type of non-invasive and non-sealing patient interfaces of a respiratory support system having one or more nasal prongs. Discussion and disclosure in relation to high flow is by way of example only.

With therapies such as nasal high flow, a flow of gas delivered to the patient can be prescribed and set. A level of positive airway pressure is generated as a result of the flow, which is dependent on a range of factors such as the weight of the patient, the set flow rate, and naris occlusion.

The positive airway pressure associated with NHF has been shown to reduce inspiratory effort and work of breathing compared to standard oxygen therapy.

Non-invasive respiratory therapies such as high flow may also be gentle on the nose and nares of the patient as high flow is an open system. As the patient interface is non-sealing, the probability of nasal trauma in the patient may be reduced. For example, the use of non-sealing patient interfaces can reduce or prevent pressure sores and/or barotrauma in a patient.

It may be desired to increase the PEEP and/or to have more predictable control of pressure delivered to the patient in high flow and similar non-sealing therapy systems. An increase of PEEP and/or greater control of pressure delivered may be beneficial in providing effective therapy. For example, maintaining an adequate level of predictable PEEP may provide the patient with support to keep the airways open.

The present disclosure relates to nasal delivery elements, such as a prong or prongs of a patient interface, an interface comprising at least one such prong, and/or systems, methods and/or apparatus including at least one such prong. In use, each prong according to the present disclosure may be inserted into a naris of the patient. When inserted into the naris, a distal end of the prong is located inside the naris of the person and a proximal end of the prong is typically located adjacent to the opening of the naris. A flow of breathable gas is delivered to the patient through an inner lumen of the prong.

When the patient exhales, a flow of exhaled gas will pass between the outer surface of the prong and the inner surface of the naris until the gas exits the naris via the naris opening. The flow of exhaled gas that flows between the outer surface of the prong and the inner surface of the naris will have an average flow direction, an average flow velocity and/or an average flow rate. In general, the average flow direction within the naris is substantially longitudinal within the naris towards the opening of the naris. Portions of the flow of exhaled air may have a higher or lower velocity than the average flow velocity. One or more portions of the flow of exhaled air may flow in a direction that is different to the average flow direction.

Each prong may be formed from the same material as the patient interface from which it extends. The prongs may be soft to mitigate risk of trauma to soft tissue in the nares. The prongs may be formed to minimise kinking of the prong, particularly when located in patient naris. The prongs may be configured to be substantially fixed in position. Protrusions on the outer surface of the prong, as described herein, may be formed from the same material as the prong. The protrusions may be substantially inflexible or at least partially rigid.

The present disclosure provides prongs for a patient interface where one or more protrusions are provided on the outer surface of the prong. A prong having at least one protrusion on its outer surface may increase the ratio of cross-sectional area of the prong to the cross-sectional area of the naris and/or may provide an increased occlusion of the naris in comparison to a prong of substantially the same shape and size, with the same internal diameter, outer diameter, prong length and wall thickness, having no protrusions.

Including at least one protrusion on the outer surface of a prong may increase the ratio of the cross-sectional area of the prong to the cross-sectional area of the naris. A prong with at least one protrusion on the outer surface may provide an increased occlusion of the naris compared to an equivalently sized prong with no protrusions on its outer surface.

The use of a prong with one or more protrusions on the outer surface may increase the resistance to flow of the exhaled gas, which may increase PEEP. Resistance to flow can be increased by, for example, increasing occlusion of the nares while maintaining a gas flow path for exhaled gas to exit and/or by creating a tortuous flow path for gas to exit the nares.

A prong having protrusions in this manner utilised with a non-invasive respiratory therapy system may provide an increase in the PEEP level provided to a patient compared to the use of a prong of the same shape and dimensions without protrusions.

The use of one or more protrusions on the outer surface of the prong may lead to an unrecoverable loss of pressure in the flow of exhaled gas, at least where the protrusion provides a sudden localised decrease in the cross-sectional area through which the exhaled gas flows. This loss of pressure in the exhaled gas can lead to an increase in PEEP. The protrusions may provide a tortuous path for at least a portion of a flow of exhaled gas and/or may result in a changing resistance to the flow. In the vicinity of the protrusions there may be a localised change of the velocity of the flow of exhaled gas. The use of one or more protrusions on the outer surface of the prong may lead to a localised slowing down of at least a portion of the exhaled gas within the naris. This may act to convert the dynamic pressure of at least a portion of the exhaled gas to static pressure. This may lead to an increase of PEEP experienced by the patient. The protrusion(s) on the prong may provide the exhaled gas with a tortuous path of exit which may result in and/or cause an unrecoverable loss in pressure that will increase PEEP. This is because the patient must generate additional pressure to account for the unrecoverable pressure loss.

According to the present disclosure, the occlusion of a naris may be increased by a prong having at least one protrusion on its outer surface compared to a prong of the same dimensions without any protrusion(s).

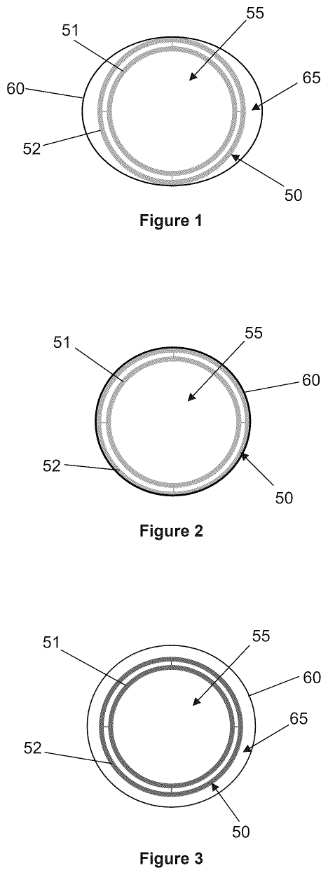

A safe gas flow path may be maintained to prevent the nare(s) from being fully occluded. FIGS. 1, 2 and 3 show representations of naris 60 cross-sections in which a prong 50 is inserted. The end view of the prong 50 is shown where protrusions 54 are in different locations along the length of the prong 50. FIG. 1 shows a non-circular nare and FIGS. 2 and 3 shows a substantially circular nare.

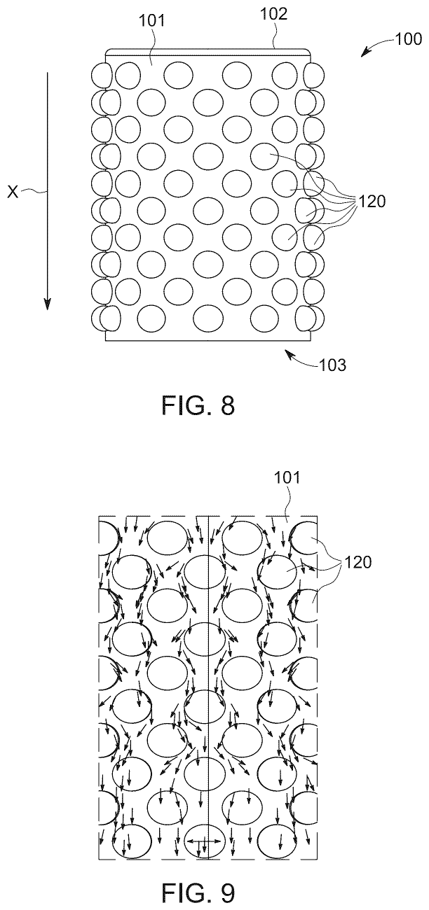

The prong 50 has a wall 51 that defines an inner lumen or inner passage 55 for the delivery of a respiratory gas to a patient. The prong 50 also has at least one protrusion 54. An outer extent 52 of the protrusion(s) 54 is spaced outwardly from the wall 51 of the prong 50. The shape and type of protrusion(s) 54 on the prong 50 is not shown in FIGS. 1, 2, 3. However, it is noted that any suitable protrusion(s) 54 may be utilised on the prong in this example. For example, the protrusion(s) 54 on the prong 50 could be according to any of the examples shown herein in FIGS. 8 to 37, or otherwise.

FIG. 3 shows an example where the prong 50, including the outer extent 52 of its protrusion(s) 54, is located inside the naris 60 without touching the inner surface of the naris 60. A space 65 is maintained between the inner surface of the naris 60 and the prong 50. The space 65 may act as a path for exhaled gases. The protrusion(s) 54 on the prong 50 increases the occlusion of the naris 60 compared to a prong of the same size which does not include protrusions 54 on its outer surface.

As shown in FIG. 1, the prong 50 including the outer extent 52 of its protrusion(s) 54 is located inside the naris 60 with the outer extent 52 of the protrusion(s) 54 touching the inner wall of the naris 60. In the example shown, the outer extent 52 touches the inner wall of the naris 60 at two locations. The prong may make contact with the nare at one location or more than two locations. A space 65 is maintained between the inner surface of the naris 60 and the prong 50 in the areas where the outer extent 52 of the protrusion(s) 54 does not touch the inner wall of the naris 60.

FIG. 2 shows an example where the outer extent 52 of the protrusion(s) 54 of the prong 50 are in contact with portions along the inner wall of the naris 60. This provides an example of an upper limit to the occlusion provided by a prong 50 having one or more protrusions 54 according to the present disclosure. The example shown in FIG. 2 is an end view and the naris 60 is not fully occluded. The protrusions 54 are in contact with the inner wall of the naris 60 at different locations along the length of the nare. A safe flow path for exhaled gas may be provided between the protrusions 54 on the outer surface of the prong 50. For example, FIGS. 8 to 37 show examples of prongs having an arrangement of protrusions 54 which maintains a safe flow path for exhaled gas along the outer surface of the prong between the protrusions.

Prongs with protrusions ensure a safe gas flow path is maintained in the event where the prongs are inappropriately sized for a patient. For example, where the cross-sectional size of the prong 50 including the outer extent 52 of the protrusion(s) 54 is greater than the internal cross-sectional size of the naris 60. Or for example, if the shape of the naris is extremely irregular and causes a mismatch with the outer surface of the prong. The inclusion of the protrusions on the outer surface of the prong presents a flow path for the gases in the nare to escape between the protrusions. Thus, a safe gas flow path is maintained for the patient.

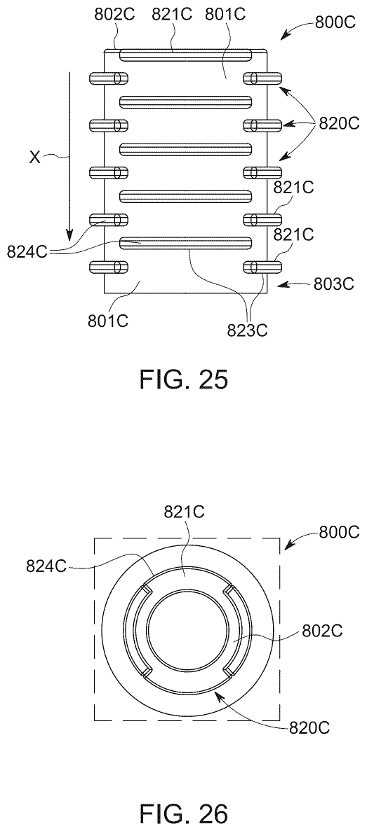

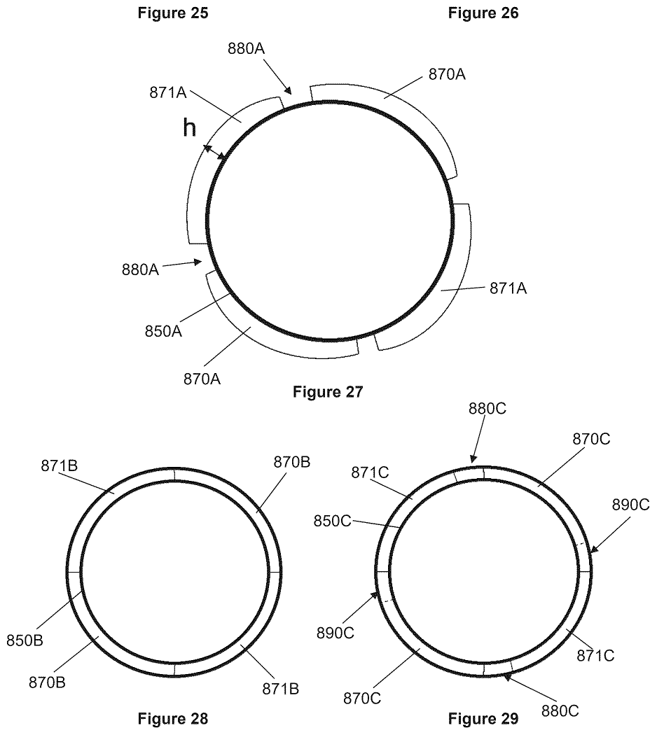

With reference to FIGS. 25 and 26, a flow of exhaled gas will travel generally in the direction X when prong 800C is located in a naris. The prong 800C includes protrusions 820C. The flow of exhaled gas will initially meet the distal end 802C of the prong 800C and a portion of the flow of exhaled gas will travel along the outer surface 801C or in close proximity to the outer surface 801C of the prong 800C generally in the direction X.

When the prong 800C is in a naris, each protrusion 820C of the prong 800C creates a localised reduction of available cross-sectional area between the outer surface 801C of the prong 800C and the inner surface of the naris in which gases may flow compared to where no protrusion is present. These locations on the prong where at least one protrusion is present may lead to localised changes in the velocity of the flow of gas. For example, there may be localised increases in velocity of the flow of the exhaled gas. Where the available cross-sectional area for a flow of gas is at a minimum, e.g. at locations where at least one protrusion is present, the velocity of the flow of exhaled gas may be at a maximum. The velocity of the flow of exhaled gas may reduce when it has passed from a location where at least one protrusion is present to a location where no protrusion is present.

When the flow of exhaled gas travelling generally in direction X meets a first surface 821C of a protrusion 800C, regions of turbulence of the flow are promoted. These regions of turbulence may have some parts of the flow of exhaled gas that are redirected and do not travel generally in the direction X. However, the average flow direction may remain in direction X. The regions of turbulence which redirect flow path of the exhaled gas or portions of the exhaled gas may lead to loss of dynamic pressure. This may result in the patient experiencing higher PEEP.

The forces that oppose the flow of exhaled gases contributes to the resistance to flow (RTF). For example, RTF may be increased by the interaction between the exhaled gas and one or more surfaces of the protrusion(s), such as the first surface 821C which changes the flow path from general direction X. Other factors that influence RTF of the flow path are surface roughness of the material and/or any curvature and/or bends and/or tortuous flow path. This example has been made with reference to FIGS. 25 and 26, however it may be applicable to any prong having at least one protrusion on its outer surface within the scope of the present disclosure.

The present disclosure includes a method of providing respiratory support to a patient. The method may comprise providing a respiratory therapy system. The respiratory system may be as substantially described herein or otherwise. The respiratory therapy system may comprise at least one of a respiratory gases source, a breathing tube to receive the respiratory gases, and a patient interface. The patient interface may be a nasal interface, such as a nasal cannula. The patient interface may have a gases inlet in fluid communication with the breathing tube to deliver the respiratory gases to a patient. The patient interface may comprise at least one prong having at least one protrusion on an outer surface of the prong. The method of providing respiratory support may further include locating the at least one prong in a naris of the patient in a non-sealing manner. The method may include operating the respiratory therapy system to provide a flow of gases to the patient interface. A flow of gases from the respiratory therapy system may be delivered to the patient through the at least one prong. At least one protrusion may provide an increased resistance to a flow of exhaled gas from the naris of the patient.

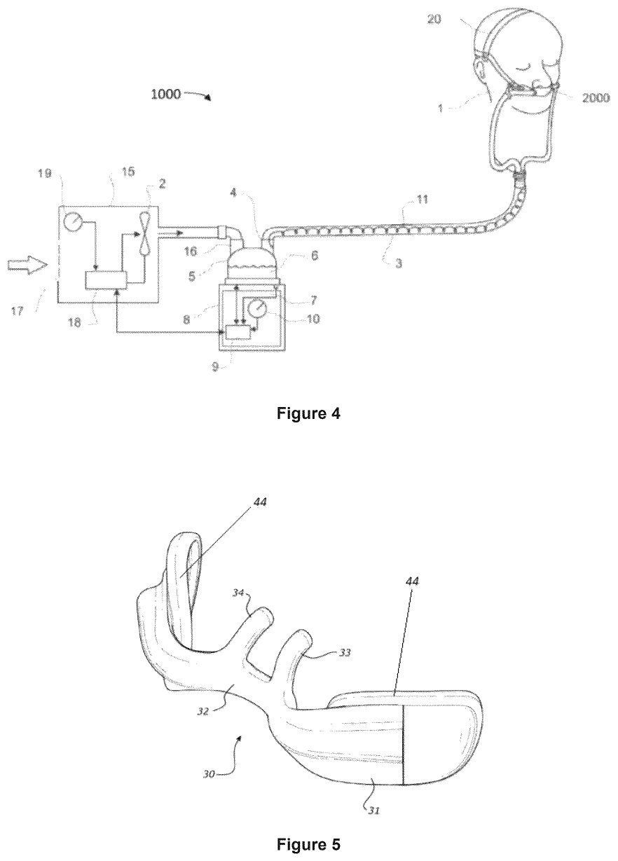

A schematic representation of an example respiratory support apparatus (or respiratory support system) that may be used with a patient interface having at least one prong according to the present disclosure is shown in FIG. 4. The respiratory support system 1000 may provide respiratory therapy or support to a patient.

The respiratory support system 100 may include a combination of components selected from, but not limited to, one or more of: a flow/gas source; a humidifier for humidifying and/or warming gas flow; conduit(s) (e.g. dry line and/or heated breathing tube); and/or a patient interface.

The respiratory support system 1000 may comprise a flow source or gas source for providing a gas, such as air, oxygen, air blended with oxygen, or a mix of air and/or oxygen and one or more other gases. The system may have a connection for coupling to the flow or gas source. The flow/gas source could be an in-wall supply, a tank and/or a flow source with a flow generator. The flow generator may have a gas inlet and may connect to an oxygen source. The flow generator can control flows delivered to the patient using one or more valves, or may comprise a blower 15, for example, as shown in FIG. 4. The flow/gas source provides a flow of gas that can be delivered to a patient, for example via an inspiratory conduit and patient interface. The flow source may provide a base gas flow rate of between about 0.5 LPM and about 375 LPM or any suitable sub-range within that range.

The blower 15 may be provided with a variable speed pump or fan 2 that draws gas or other gases through a blower inlet 17. The speed of the variable speed pump or fan 2 may be controlled by a control means or electronic controller 18 in response to inputs from a controller 9 and a user-set predetermined value (preset value) of pressure, flow rate and/or fan speed via one or more input devices 19. The function of the electronic controller 18 may be carried out by the controller 9.

The patient interface may be an unsealed (non-sealing) interface. The patient interface comprises at least one prong for insertion into the nare(s) of a patient or user to deliver a flow of gas to the patient/user.

A humidifier may be provided between the flow source and the patient to humidify and/or warm the delivered gas. The humidifier may be controlled by a controller. Various humidifier configurations may be employed. The humidifier may comprise a humidification chamber. The humidification chamber may be removable, for example, may be partially or entirely removable or disconnected from the flow path and apparatus.

The humidification chamber may comprise a gas inlet and a gas outlet for connection into the gas flow path of the apparatus/system. For example, flow of gases from the flow generator is received into the humidification chamber via the gas inlet and exits the chamber via the gas outlet after being heated and/or humidified. The humidification chamber may contain a volume of liquid, typically water or similar. In operation, the liquid in the humidification chamber is controllably heated by one or more heaters or heating elements associated with the chamber to generate water vapour or steam to increase the humidity of the gases flowing through the chamber.

In one configuration, the humidifier is a pass-over humidifier.

In one configuration, the humidifier may comprise a heater plate, for example associated or within a humidification bay that the chamber sits on. The chamber may be provided with a heat transfer surface, such as a metal insert, plate or similar, in the base or other surface of the chamber that interfaces or engages with the heater plate of the humidifier.

The humidification chamber may be any suitable shape and/or size. The location, number, size, and/or shape of the gas inlet and gas outlet of the chamber may be varied as required. In one configuration, the humidification chamber may have a base surface, one or more side walls extending up from the base surface, and an upper or top surface. The gas inlet and gas outlet may be positioned on the same side of the chamber. In another configuration, the gases inlet and gases outlet may be on different surfaces of the chamber, such as on opposite sides or locations. The humidification chamber 5 may be formed from a plastics material and may have the heat transfer surface referred to above (for example an aluminium base) which may be in direct contact with a heater plate 7 of the humidifier 8. The humidifier 8 may be provided with a control mechanism or electronic controller 9, such as a microprocessor based controller, executing computer software commands stored in associated memory.

An inspiratory conduit 3 may be coupled to a gas outlet of the respiratory support system 1000 at a first end and may be coupled to the patient interface 2000 at a second end. A heating element 11 may be provided within the inspiratory conduit 3 to help prevent condensation of the humidified gases within the conduit 3. The heating element 11 in the inspiratory conduit 3 may be controlled by a controller.

The inspiratory conduit 3 and/or a tube of the patient interface may comprise a breathable material. For example, at least part of the wall of the inspiratory conduit/tube and/or tube may comprise the breathable material. The breathable material may permit passage of water vapour without allowing bulk passage of liquid water or bulk flow of respiratory gases therethrough. A breathable material may assist to reduce condensate within the tube or inspiratory conduit.

In the example shown in FIG. 4, the patient interface 2000 is a nasal cannula that is supplied with gas from a flow/gas source, which in this example is blower 15. The patient interface 2000 may include headgear 20 to support and retain the patient interface on the patient in a position suitable for delivery of therapy.

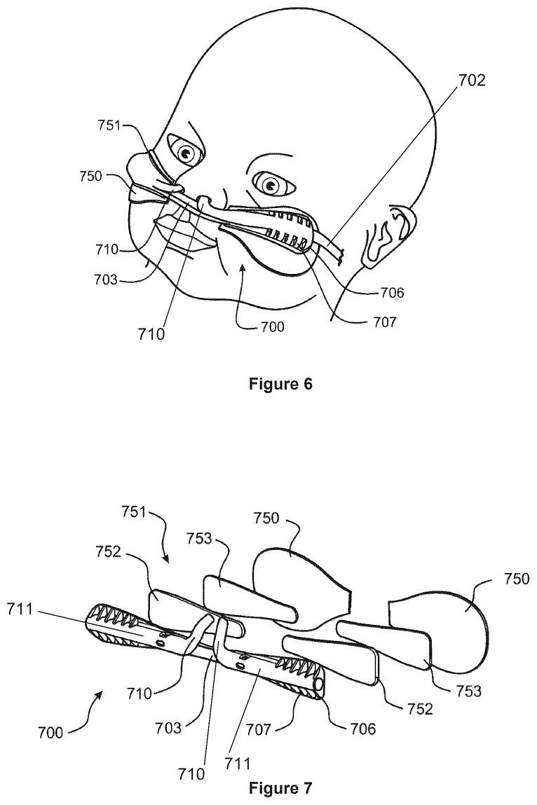

Other means for retaining a patient interface on the patient may be used in place of or in addition to the headgear 20. For example, the patient interface may be attachable to the patient via adhesive elements. For example, a two-part releasable securement assembly 751 as shown in FIGS. 6 and 7 may be used.

As shown in FIG. 4, for example, the patient interface 2000 may be connected to a humidified gas transportation flow path or inspiratory conduit 3. The inspiratory conduit 3 may be connected to an outlet 4 of humidifier 8, including a humidification chamber 5, that is supplied with breathable gases. The gas or gases can be supplied from a source that is external to and/or separate from the respiratory support system 1000, or from a source that is internal to and/or integrated with the respiratory support system 1000. The system may have an intermediate conduit between the outlet 4 of the humidification chamber 5 and the inspiratory conduit 3.

The controller 9 may receive an input from an input device 10, through which a user may set a predetermined required value (preset value) of humidity or temperature of the gas supplied to the patient. In the example shown in FIG. 4 the input device 10 is a dial, but any suitable user input device may be used. In response to the user-set humidity or temperature value input and other possible inputs such as internal sensors that sense gas flow or temperature, or other parameters, the controller 9 determines when (or to what level) to energize the heater plate 7 to heat the water 6 within the humidification chamber 5. As mentioned above, humidification of the gases provided to the patient is an optional feature and respiratory support devices are possible that do not include apparatus for humidifying the gas.

FIG. 5 shows a front perspective view of an example patient interface in the form of a nasal cannula 30 comprising a pair of prongs 33,34. The prongs are configured for insertion into the nares of a patient. According to the present disclosure, one or both prongs 33,34 may comprise at least one protrusion on its outer surface (not shown in FIG. 5). The prongs 33, 34 may be configured such that at least one prong does not fully occlude the patient nares. The prongs 33, 34 may provide a safe passageway for exhaled gas to escape around each protrusion and/or along the outer surface of each prong.

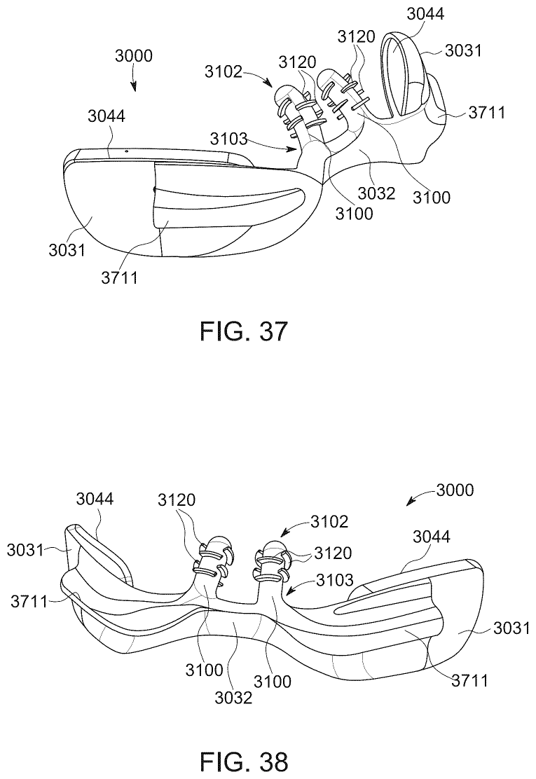

FIGS. 37 and 38 show a similar patient interface/nasal cannula 3000 to that of FIG. 5 but having protrusions 3120 according to the present disclosure on the outer surface of each of the prongs 3100. The prongs 3100 may have any desired number of protrusions 3120 on their outer surface. The protrusions 3120 may be of any size or shape. For example, the protrusions 3120 may be as disclosed with reference to any one or more of the FIGS. 8 to 36, or as otherwise described herein.

In FIGS. 37 and 38 the protrusions 3120 are circumferentially elongate protrusions. The protrusions 3120 may be arranged in rows circumferentially about the prongs 3100. The protrusions 3120 of one row may be offset or staggered relative to the protrusions 3120 of each adjacent row.

Each prong 3100 extends from a proximal end 3103 to a distal end 3102. The proximal end 3103 of each prong 3100 is connected to the body of the nasal cannula 3000. The distal end 3102 of each prong 3100 in these examples, includes an outlet. Each prong 3100 in this example includes a curved shape between its proximal end 3103 and its distal end 3102. Each prong 3100 may be a curved prong.

One or both prongs 3100 may have a cross-sectional area that changes from the proximal end 3103 to the distal end 3102. The protrusions as described herein may be applied to any size or shape of prong, including curved prongs as shown in FIGS. 37-40.

Each prong may include an inner passage or lumen. The inner passage/lumen extends between the proximal end 3103 and distal end 3102 of the prong 3100. The nasal cannula 3000 may have cannula conduits 3711 which are in fluid communication with the inner passage/lumen of prongs 3100. A breathable gas may be delivered through the cannula conduits 3711 to the inner passage/lumen of the prongs 3100 and exiting via the outlet at the distal end 3102.

A connector (not shown) may be used to connect a cannula conduit 3711 to a gas delivery tube or inspiratory conduit. The cannula conduits 3711 may connect to a respective gas delivery tube or inspiratory conduit. The inner passage/lumen of prongs 3100 may be in fluid communication with a respective one of the cannula conduits 3711. In the example shown in FIGS. 37 and 38 each prong 3100 is in fluid communication with the cannula conduit 3711 on the respective adjacent side of the nasal cannula 3000.

In other examples, the prongs 3100 may each be in fluid communication with both cannula conduits 3711. For example, a conduit within the central portion 3102 of the nasal cannula 3000 may fluidly connect the cannula conduits 3711 on either side.

According to some examples, the nasal cannula may comprise a single cannula conduit 3711. The single cannula conduit 3711 may supply breathable gas to the inner passage/lumen of both prongs 3100. The nasal cannula 3000 may comprise a manifold in fluid communication with the prongs 3100. The prongs 3100 may be in fluid communication with a common gas delivery tube or inspiratory conduit. The cannula conduits 3711 may be in fluid communication with a common gas delivery tube or inspiratory conduit.

It is noted that the present disclosure relates to the use of one or more protrusions on the outer surface of at least one prong of a patient interface. This is not restricted to use of any specific type of interface, such as those shown in FIGS. 5 to 7, 37, 38 and 39. At least one protrusion may be provided on at least one prong of any patient interface that comprises at least one prong. This includes patient interfaces that have a pair of nasal prongs that are supplied with gas from a common conduit, and which may have a manifold in fluid communication with both prongs. The present disclosure includes patient interfaces that have a pair of prongs, with each prong supplied with gas from separate conduits. The separate conduits may meet at a common connection point for connection to inspiratory conduit.

The patient interface may be suitable to deliver a gas flow to the patient's nasal cavity/nares at a pressure that is predictable for a given or set flow rate. Providing the gas flow to the patient's airway may include providing the gas flow at any suitable flow rate. The patient interface may deliver a flow of gases to the patient over a wide range, for example about 0.5 LPM (litres per minute) or higher, depending on therapy/respiratory support and/or patient type.

Without limitation, delivery of gases to a patient can be from about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, a flow rate of gases supplied or provided to an interface via a system or from a flow source or flow modulator, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

Flow rates for premature/infants/paediatrics (with body mass in the range of about 1 to about 30 kg) can be different. Without limitation, the flow rate can be set to 0.4-8 LPM/kg with a minimum of about 0.5 LPM and a maximum of about 70 LPM. For patients under 2 kg maximum flow may be set to 8 LPM.

The gas delivered can be chosen depending on for example, the intended therapy and/or respiratory support. Gases delivered may comprise a percentage of oxygen (also referred to herein as fraction of oxygen). In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

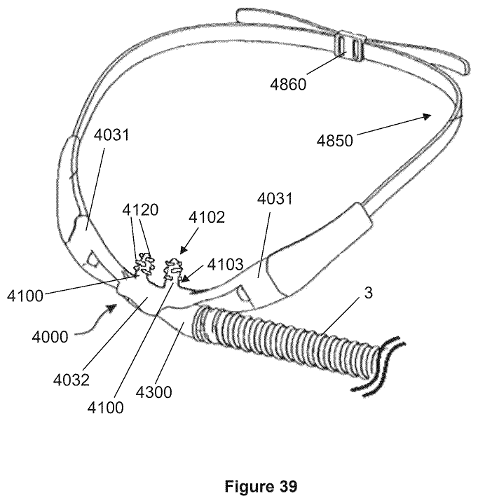

FIG. 39 shows a perspective view of another example of a patient interface according to the present disclosure. The patient interface of this example is a nasal cannula 4000 with a common gas manifold. The nasal cannula 4000 has a body with a central portion 4032. The body of the nasal cannula 4000 includes arms or wings 4031. The arms or wings 4031 extend from either side of the central portion 4032. The nasal cannula has a pair of prongs 4100. Each prong 4100 extends from a proximal end 4103 to a distal end 4102. The prongs 4100 extend substantially from the central portion 4032 of the body of the nasal cannula 4000.

The patient interface may be head mounted. The patient interface may be affixed to a person through adhesive. The patient interface may comprise at least one arm that is attachable to a headgear and/or at least one strap. Other methods of attachment of the patient interface to the patient may also be utilised.

In the example shown in FIGS. 5, 37 and 38, the nasal cannula 30, 3000 comprises a body 32, 3032 having a central section and a pair of side arms 31, 3031 on either side of the body 32, 3032. The prongs 33,34, 3100 extend from the central section of the body 32, 3032. A patient securement assembly may comprise one or more facial pads 44, 3044 located on the side arms 31, 3031. During use, the facial pads 44, 3044 may be removably attached to or lie adjacent the patient's cheeks. In some configurations, the facial pads 44, 3044 may have an adhesive surface that allows the facial pads 44, 3044 to be removably attached to the patient's cheeks. In other configurations, the facial pads 44, 3044 may attach to one or more dermal patches, as described further. In certain scenarios this may be more comfortable for the patient, and may reduce the chance of the nasal cannula 30, 3000 shifting from its correct position during use.

In other configurations, the nasal cannula may be attached to the patient's head via one or more straps or via a headgear, such as shown in FIG. 4 or 39. The nasal cannula 4000 of the example of FIG. 39 includes a strap 4850. The strap 4850 may be used to retain the nasal cannula 4000 against the patient's face. The strap 4850 may extend around the head of the patient.

In the example shown, the strap 4850 includes an adjustment buckle 4860. The adjustment buckle 4860 may be manipulated by pulling on the appropriate portion of the strap 4850 to adjust its effective length. Adjustment of the buckle 4860 alters the length of the loop of the strap 4850 to tighten or loosen the strap 4850 in use. Other methods for tightening or loosening the strap 4850 may be utilised. For example, the strap may utilise an adhesive, which may be releasable and reattachable, or hook and loop material, or any other appropriate means or mechanism.

Each of the prongs 33, 34 can have a notional central axis that runs substantially longitudinally through the centre of the lumen of each of the prongs 33, 34 from the base to the tip. During use, the central axis of each prong 33, 34 may be parallel to the direction of the gases flow within that prong.

Within any of the examples described herein, the prongs may have a substantially circular cross-section. The prongs may have a non-circular cross-section. The prongs may have a substantially elliptical or oval cross-section. The cross-sectional shapes referred to here may apply to the cross-sectional shape of the prong at the outlet and/or at the base (where gas enters the prong), and/or at any position along the length of the prong.

The prongs may have a cross-section that has at least one flat edge, when said cross-section is taken perpendicular to the central axis defined above. For example, the cross-section may be a shape with entirely flat edges, such as a rectangle or a triangle. The cross-section may be a shape with a mix of one or more curved edges and at least one flat edge, such as a semicircle. This flat edge results in a flat surface along one face of each prong.

In some configurations, the cross-section is consistent throughout the length of each prong. In other configurations, the size and/or dimensions of the cross-section changes throughout the length of each prong. For example, each prong may taper inwards along its length. In further configurations, the prongs do not have a consistent cross-sectional shape.

FIGS. 6 and 7 show an example of a patient interface which is a nasal cannula 700. In this example, the nasal cannula 700 broadly comprises a body 703, a pair of prongs 710, at least one gases inlet conduit 702, a connector (not shown) and securement assembly 751. The securement assembly 751 enables a user to place and maintain the nasal cannula 700 in the correct operational position. The gases inlet conduit 702 forms a fluid or gases connection between the outlet end of the inspiratory conduit and the nasal cannula 700 to allow fluids or gases to flow between the inspiratory conduit and nasal cannula 700. The connector may be, in use, connected to and in fluid communication with the gases inlet conduit 702. The connector may removably attach the gases inlet conduit 702 to the nasal cannula 700.

As shown in FIGS. 6 and 7, the patient interface 700 includes a pair of prongs 710 extending from a central portion of the patient interface 700. Gases flow may pass through at least one cannula conduit 711 on the body 703 to the prongs 710 for delivery to the patient. In some configurations, the prongs 710 may each have an independent flow path, for example cannula conduits 711 as shown in FIGS. 6 and 7. Each prong 710 may have separate delivery tubing. The delivery tubing/gases inlet conduit may be fluidly connected at the connector end of the interface.

In other configurations, the prongs 710 may be in fluid communication with each other, such as via a manifold as shown in FIG. 39. The manifold may be in fluid communication with the gases inlet conduit 702. As shown in FIG. 39, the manifold may be in the central portion 4032 of the body of the nasal cannula 4000. The manifold may be in fluid communication with each of the prongs 4100. The manifold may be in fluid communication with one or more gas delivery conduits 3. A connector 4300 may be used to fluidly connect a gas delivery tube or inspiratory conduit 3 to the nasal cannula 4000. The connector 4300 may connect the gas delivery tube or inspiratory conduit 3 to the manifold.

With continued reference to FIGS. 6 and 7, the body 703 of the patient interface 700 comprises a pair of wings 707 located at opposed ends 706 of the body 703. At least one wing 707 may comprise a cannula conduit 711 which fluidly connects one or both prongs 710 to a tube/gases inlet conduit for delivering a breathable gas. In other examples, one or both wings 707 may comprise a cannula conduit that fluidly connects a manifold to the tube/gases inlet conduit. One or both wings 707 may function as at least portions of side straps of the patient interface (not shown in FIGS. 6 and 7). One or both wings 707 may at least partially support a cannula conduit. One or both of the prongs 710 may have at least one protrusion on its outer surface (not shown in FIGS. 6 and 7).

The patient interface 700 of FIGS. 6 and 7 may be attachable to one or more fixation structures or dermal patches including a body 750 and fixing element 753. The patient interface 700 may include a surface on the patient facing side of each wing 707 to which an interface attachment element 752 may be attached. The interface attachment element 752 has a patient facing side and an interface facing side. The interface facing side of the interface attachment element 752 is attachable or affixed to the patient interface 700, such as by an adhesive, for example. The interface attachment element 752 may be integrated with or suitably adhered to the patient interface 700.

The body 750 of each fixation structure has a patient side that faces the patient's skin and an interface side that faces the patient interface 700. The interface side of the body 750 is provided with or otherwise adhered to a fixing element 753. The fixing element 753 is the first part of a two-part releasable securement assembly 751. A second part of the two-part releasable securement assembly 751 is the interface attachment element 752. The patient facing side of the interface attachment element 752 is attachable to the interface facing side of the fixing element 753. The releasable securement assembly 751 may releasably connect the or each fixation structure with the patient interface 700.

The two-part releasable securement system 751 may comprise complementary fastening elements. For example, the two-part releasable securement system may comprise a mechanical fastener, such as a hook and loop material (such as Velcro™), a magnet or an array of magnets disposed respectively on each of the fixation structure(s) and patient interface 700 having the poles suitably arranged, an adhesive arrangement that may be activated when the two parts are brought together, or any other suitable releasable coupling. The interface side of the fixation structure 750 may have one of a hook or a loop material, and the patient side of the interface attachment element 752 may have the other of the hook or loop material, such that the fixation structure 750 and interface attachment element 752 are releasably attachable to each other.

The body 750 of the fixation structure may be releasably adhered or otherwise releasably attached to the patient's skin. The patient side of the fixation structure body 750 may be attached to the skin of a patient by a dermatologically sensitive adhesive. The adhesive may include any of: a hydrocolloid-based adhesive material; a zinc oxide-based adhesive material; a silicone-based adhesive material; a polyurethane; and/or a hydrogel-based adhesive material.

The above description in reference to FIGS. 6 and 7 is for example purposes and is non-limiting to the scope of the present disclosure. For example, the present disclosure is equally applicable to other configurations of nasal cannula or other patient interfaces, such as those that are attached to a patient via headgear and/or at least one strap, for example as shown in FIG. 4 or 39. For example, a patient interface may comprise at least one arm that is attachable to a headgear and/or at least one strap. Other methods of attachment of the patient interface to the patient are also possible within the present disclosure.

An example of a prong 100 having protrusions 120 on its outer surface 101 is shown in FIGS. 8 and 9. The prong 100 has a distal end 102 and a proximal end 103. In reference to a prong within the present disclosure, the term distal or distal end is intended to refer to the terminal end or tip of the prong, while the term proximal or proximal end is intended to refer to the inlet end or base of the prong. In use, gas flow may enter a prong at the proximal end or inlet end or base and flow towards and out of the distal end or terminal end or tip. The prong 100 has an internal passage, or lumen, between the proximal end 103 and the distal end 102. A breathable gas may be delivered to a patient through the internal passage. The patient interface may have two prongs 100. The breathable gas may be delivered to the patient interface via at least one tube from a respiratory support device, for example as described in relation to FIG. 4. When the patient exhales, an exhaled gas flows generally in the direction X as shown in FIG. 8.

One or more protrusions may have an outwardly curved shape, an outwardly rounded profile or may be dome-shaped. One or more protrusions may be at least partially rounded or curved. As shown in the example of FIGS. 8 and 9, the outer surface 101 of the prong 100 has a plurality of protrusions 120. In this example, each protrusion 120 has a substantially hemispherical shape outwardly from the outer surface 101. The surface of each substantially hemispherical protrusion 120 is convex. Each protrusion 120 creates a substantially circular footprint on the outer surface 101. Other, generally outwardly curved shaped protrusions may be provided.

The protrusions 120 are provided on the outer surface 101 in a plurality of rows that extend circumferentially about the prong 100. The protrusions 120 may be provided in line with one another between the rows. In the example shown in FIG. 8, the protrusions 120 in one row are staggered and offset relative to protrusions 120 in each adjacent row. Other formations of the protrusions 120 on the outer surface 101 of the prong 100 are also possible within the scope of the present disclosure.

FIG. 9 shows an airflow representative of a flow of exhaled gas over a section of the outer surface 101 of the prong 100. As shown, the protrusions 120 may cause the flow to undergo a plurality of direction changes between the distal end 102 and the proximal end 103 of the prong. At least a portion of the exhaled gas may be directed to traverse a tortuous path over and/or between the protrusions 120 over the outer surface 101 of the prong 100. This change of direction and/or the tortuous path followed by the exhaled gas may create localised changes to the velocity of the gas flow. The change and direction and/or the tortuous path may create a changing resistance to the flow of exhaled gas. This may lead to an increase in PEEP.

The protrusions 120 take up space outside of the prong 100, and when the prong 100 is inserted into a naris, each protrusion 120 will create a reduction in the available cross-sectional area between the outer surface 101 of the prong 100 and the inner surface of the naris compared to where no protrusion is present. The at least one protrusion 120 on the outer surface 101 of a prong 100 may increase the ratio of the cross-sectional area of the prong to the cross-sectional area of a naris in which the prong is inserted. A prong 100 with at least one protrusion 120 on the outer surface 101 may provide an increased occlusion of the naris compared to an equivalently sized prong with no protrusions. A flow of exhaled gas may travel over and/or around the protrusions 120 before exiting the naris.

A prong 100 having curved, rounded or substantially hemispherical protrusions 120 as shown in FIG. 8 may generate a higher mean patient pressure compared to a prong of equivalent dimensions having a smooth outer surface with no protrusions. The protrusions 120 on the outer surface 101 of the prong 100 provide a higher naris occlusion compared to a standard prong of the same size and may provide a higher PEEP for a given flow rate.

As a non-limiting example, a prong 100 with protrusions such as those shown in FIG. 8 may have an internal diameter of about 9.8 mm. Protrusions 120 may extend about 0.5 mm outwardly from the surface of the prong. When the prong 100 is in a naris, the prong 100 may provide around 50-70% naris occlusion. The naris occlusion provided by a prong having one or more protrusions may be, for example, 90% or higher. The naris occlusion is intended to be less than 100% to ensure that there is a safe exit flow path for a flow exhaled gas. A safe exit flow path may also be provided between the protrusions 120.

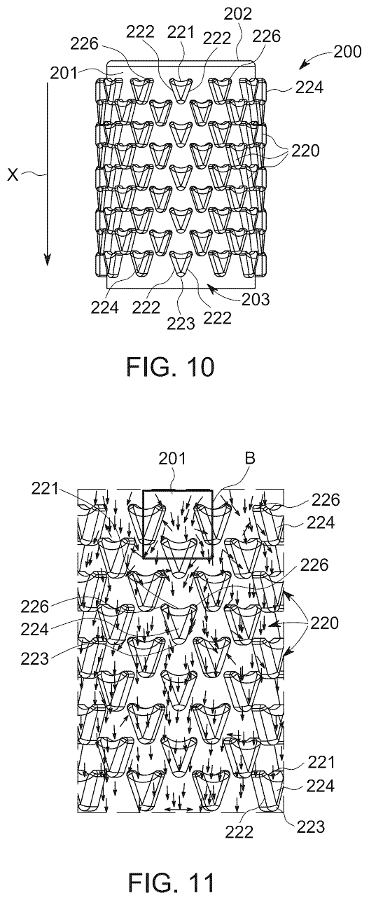

FIGS. 10 to 12 show another example of a prong 200 according to the present disclosure having a plurality of protrusions 220. The example shown in FIGS. 10 to 12 has similar features to the example described in relation to FIGS. 8 and 9, except that the protrusions 220 in this example are of a different type having a different shape and size.

Each protrusion 220 is shaped to redirect a portion of the flow of exhaled gas back against the oncoming flow. The protrusions 220 provide a tortuous path for at least part of the flow of exhaled gas. The example shown in FIGS. 10 to 12 may provide a resistance to flow of portions of the exhaled gas to provide an associated increase in PEEP.

The protrusions 220 shown in FIGS. 10 to 12 each have a first surface 221 that is substantially opposed to flow direction X. In the example shown, the first surface 221 is substantially concave relative to the distal end 202 of the prong 200. In other words, the first surface 221 of each protrusion has circumferentially spaced apart side edges 226 between which the first surface 221 is curved towards the proximal end 203. Side walls 222 each extend from the respective side edge 226 of the first surface 221 in a substantially proximal direction. The side walls 222 are angled inwardly towards one another. The side walls 222 meet one another at end 223 located in the proximal direction compared to the first surface 221. End 223 may be curved. The side walls 222 may meet at an angled point.

Protrusion 220 has an outer surface 224 between the first surface 221, side walls 222 and end 223. Protrusion outer surface 224 is spaced from the prong outer surface 201 by a height of the protrusion 220. The height of the protrusion 120 may be constant across protrusion 120. The height of the protrusion 120 may differ at different points on the outer surface 224. For example, the protrusion 120 may have a height ‘y’ adjacent to the first surface 221 and a height ‘z’ adjacent to the end 223. The height ‘y’ may be the same as the height ‘z’, the height ‘y’ may be greater than the height ‘z’, or height ‘y’ may be less than the height ‘z’. The outer surface 224 is substantially flat as shown in FIGS. 10 to 12. The outer surface may be curved or may comprise at least one curved portion.

The arrangement of protrusions 220 on the prong 200 shown in FIGS. 10 to 12 may create a tortuous path for a gas flow and/or resistance to flow. The protrusions 220 on the prong 200 may provide at least 50%, approximately 50-70%, at least 60%, at least 70%, at least 80%, less than 100%, less than 95%, or less than 90% naris occlusion. The actual occlusion provided will vary depending on the size of the naris into which the prong 200 is inserted, prong lumen diameter and/or height of protrusion(s).

The protrusions, however, maintain an open flow path, or safe exit flow path, for exhaled gas over the outer surface 201 of the prong 200 between the distal end 202 and the proximal end 203. The open flow path between the protrusions 220 is intended to be in addition to any inherent gap between the prong 200 and the inner surface of a naris, by virtue of the prong 200 and hence the interface being non-sealing, as with all examples disclosed herein.

The protrusions 220 are arranged in circumferential rows with the protrusions 220 of one row being staggered relative to the protrusions 220 of each adjacent row. It is also possible that the protrusions 220 may be arranged in manner other than that shown in FIG. 10. For example, the protrusions may not be arranged in circumferential rows or may be arranged in a substantially random pattern over the outer surface of the prong 200.

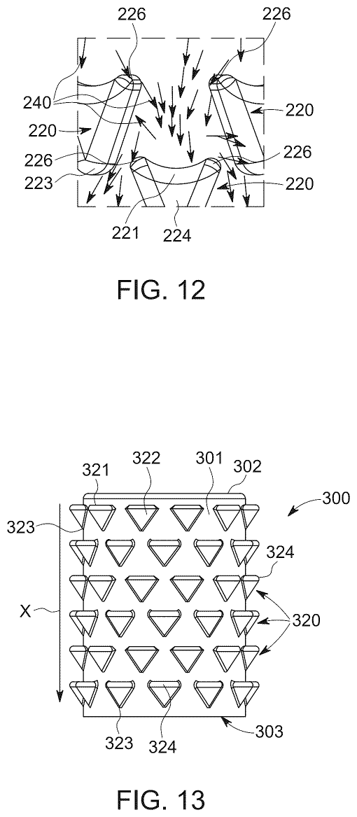

The protrusions 220 redirect portions of the exhaled gas back towards exhaled gas travelling in the direction X. Some portions of the exhaled gas may be redirected in a substantially circumferential direction by the first surface 221 of the protrusions 220. FIGS. 11 and 12 show possible flow directions of portions 240 of the flow of exhaled gas. FIG. 12 is an enlarged image of section B of FIG. 11. As shown in these Figures, some portions 240 of the flow of gas are redirected by the first surface 221 of protrusion 220. Portions 240 of the flow of exhaled gas may be redirected in one or more directions.

The first surface 221 of each protrusion 220 provides resistance to flow that may assist in slowing down portions of the exhaled gas. In order to maintain an average velocity of the flow, other portions of the exhaled gas may increase in velocity. The prong 200 may therefore increase PEEP compared to a prong of equivalent dimensions without any protrusions for a given flow rate.

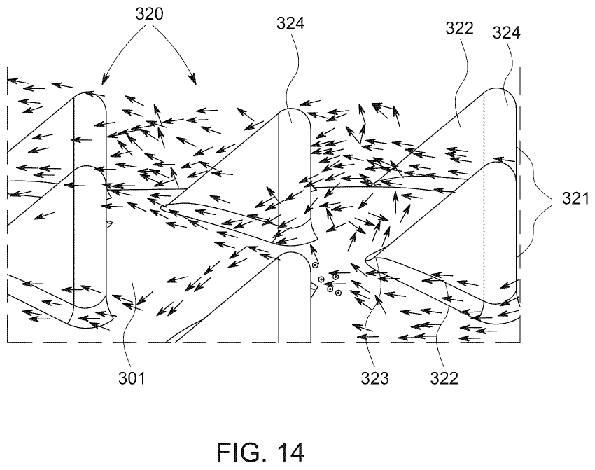

FIGS. 13 and 14 show a further example of a prong 300 having protrusions 320 according to the present disclosure. These examples have the same or similar features as prongs shown in other examples, such as in FIGS. 8 to 12, except that the protrusions 320 are of a different type having a different shape and size.

The prong 300 comprises a plurality of protrusions 320. Each protrusion 320 is substantially coned shaped. Each cone shaped protrusion 320 has a first surface 321. The first surface 321 is located on the distal side of the protrusion 320. In other words, the first surface 321 generally faces towards the exhaled flow.

The first surface 321 may be substantially perpendicular to the outer surface of the prong 301. The first surface 321 may be substantially perpendicular to the direction X. The first surface 321 may be a straight wall and/or the first surface 321 may be substantially flat. Additionally or alternatively, the first surface 321 may be curved or may comprise a curved section. In some examples, the first surface 321 may be inwardly curved, outwardly curved, inwardly angled, outwardly angled, or angled relative to the circumference of the prong 300.

The first surfaces 321 of all the protrusions 320 may be substantially the same shape and/or alignment. The shape and/or alignment of the first surfaces 321 may vary between protrusions 320.

As shown in FIG. 14, the protrusion 320 may comprise a lip 324 about the edge of the first surface 321. The lip 324 may be curved. The lip 324 may be an angled edge. From the lip 324 or edge of the first surface 321, a cone wall 322 extends substantially in the proximal direction towards the point 323.

The cone shaped protrusions 320 may be provided on the prong 300 in a plurality of rows. The rows may be arranged circumferentially about the prong 300. In the example shown in FIGS. 13 and 14, the protrusions 320 of one row are staggered and offset relative to the protrusions 320 of each adjacent row. The staggered pattern of protrusions 320 on the outer surface 301 of the prong 300 creates a winding and/or tortuous flow path for exhaled gas to follow. This arrangement of protrusions 320 may assist in decreasing the dynamic pressure of exhaled gas and increasing static pressure.

A possible flow of exhaled gas over the outer surface 301 of the prong 300 and around the cone shaped protrusions 320 is shown in FIG. 14. The first surface 321 slows and/or redirects the exhaled gas flow. For example, the redirection of the gas flow creates mixing of the exhaled gas and increases turbulence. Eddies may be induced around the protrusions 320. The induced eddies may assist in slowing down portions of the exhaled air. This also leads to an increase in PEEP in the airway of the patient in use, compared to the use of the prong of comparable size without any protrusions for a given flow rate.

According to the examples provided herein, portions of the flow of exhaled gas in direction X may interact with at least the first surfaces 221, 321, 421, 521, 621, 821, 841B of the protrusions 220, 320, 420, 520, 620, 820, 840B within the naris. The protrusions reduce the cross-sectional area between the outer surface of the prong and the inner surface of the naris. The protrusions increase occlusion of the naris compared to a prong of equivalent dimensions which does not have any protrusions. The prongs according to the present disclosure provide occlusion of less than 100% to maintain an open flow path for exhaled gas to exit the naris. The prong having protrusions according to the present disclosure may provide about 50-70% occlusion of the naris. The prong with one or more protrusions may provide occlusion of about 40-90% of the naris. The protrusions on the prong may result in a higher expiratory pressure within the naris compared to a prong of equivalent dimensions with no protrusions.

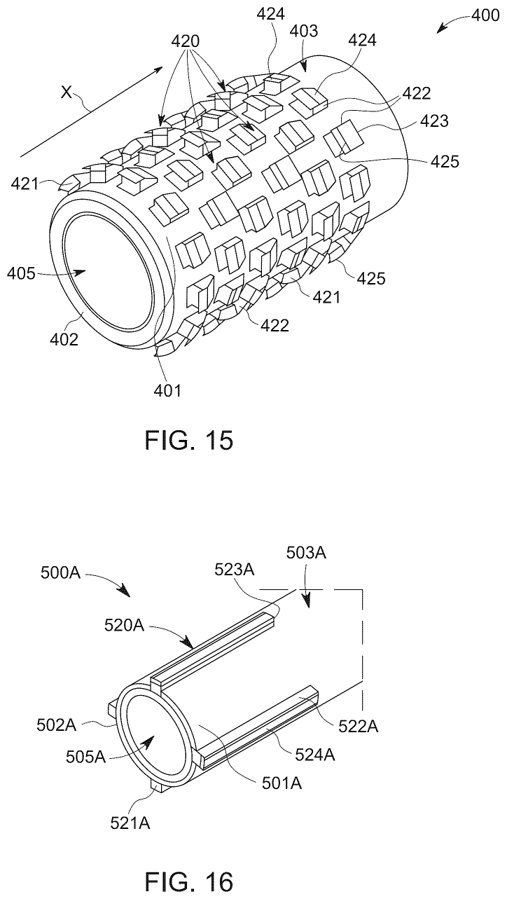

FIG. 15 shows another example of a prong 400 having protrusions 420 according to the present disclosure. The example prong 400 shown in FIG. 15 has the same features as the prongs shown in FIGS. 8 to 14, except that the protrusions 420 in this example are of a different type having a different shape and/or size than the previous examples described. The prong 400 has an opening 405 at its distal end 402.

The prong 400 includes a plurality of protrusions 420 on its outer surface 401. The protrusion 420 has a first surface 421 located on the distal side of the protrusion 420. In other words, the first surface 421 faces generally towards the exhaled flow.

The first surface 421 may be substantially perpendicular to the outer surface 401 of the prong 400. The first surface 321 may be substantially perpendicular to the direction X.

The first surface 421 as shown in FIG. 15 includes a curved face. The curved face is concave relative to the distal end 402 of the prong 400. The first surface 421 is curved proximally away from the outer surface 401 of the prong 400 until it reaches a base of the curve. From the base of the curve the first surface 421 continues to be curved distally away from the outer surface of the prong until it reaches the edge 425 of the first surface 421.

The first surface 321 may be inwardly curved, outwardly curved, inwardly angled, outwardly angled, or angled relative to the circumference of the prong 300. The first surface 421 may be planar. The first surface 421 may be convex or concave.

The first surfaces 421 of all of the protrusions 420 on a given prong may be substantially the same shape and/or alignment. The shape and/or alignment of the first surfaces 421 may vary between protrusions 420 on a given prong.

Side walls 422 on either side of the protrusion 420 extend in a proximal direction from the sides of the first surface 421. The side walls 422 of each respective protrusion 420 may be substantially parallel to one another, as shown in FIG. 13. The side walls 422 may be substantially parallel to direction X. The side walls 422, may be planar or may be curved.

In some examples, the side walls 422 may be angled inwardly towards one another. For example, the side walls 422 may be angled inwardly towards one another and meet at a proximal edge 423. The side walls 422 may be angled inwardly towards one another and towards the proximal edge 423, without meeting. The side walls 422 may extend such that the first surface 421 has a width that is greater than a width of the proximal edge 423.

In some examples, the side walls 422 may be angled outwardly away from one another towards the proximal edge 423. The side walls 422 may extend such that the width of the first surface 421 is less than the width of the proximal edge 423.

Outer wall 424 extends from an edge 425 of the first surface 421 to the proximal edge 423. The proximal edge 423 may be on or adjacent the outer surface of the prong. Each side of the outer wall 424 of the protrusion 420 meets a respective side wall 422. The outer wall 424 may be outwardly curved, as shown in FIG. 15.

In some examples, the outer wall 424 may be planar. The outer wall 424 may be inwardly curved, rather than outwardly curved as shown in FIG. 15.

In the example shown in FIG. 15, the protrusions 420 are provided in a plurality of rows. The rows are arranged circumferentially about the prong 400. In the example shown in FIG. 15, the protrusions 420 of one row are staggered and offset relative to the protrusions 420 of each adjacent row. The staggered pattern of protrusions 420 on the outer surface 401 of the prong 400 creates a winding and/or tortuous flow path for exhaled gas along the outer surface 401 of the prong 400. The staggered pattern of protrusions 420 may increase the surface area the exhaled gas meets, which may further increase resistance to flow. This arrangement of protrusions 420 may assist in decreasing the dynamic pressure of exhaled gas and increasing static pressure.

In some examples, the protrusions 420 of one row may be substantially parallel to the protrusions 420 of one or more other row and not staggered relative to one another. The protrusions 420 may be aligned with one another between rows and arranged in columns. The protrusions 420 of each row may be circumferentially parallel with protrusions 420 of each adjacent row. The protrusions 420 of one row may be partially offset in alignment relative to the protrusions 420 of one or more other row. The protrusions 420 of one row may be partially offset in alignment relative to the protrusions 420 of each adjacent row.

The concave/curved shape of the first surface 421 of each protrusion 420 may redirect gas flow into the exhaled flow. At least a portion of gas meeting the first surface 421 may be redirected towards the oncoming flow of exhaled gas. Redirected flow may increase turbulence. The protrusions 420 may increase resistance to flow of the gas path for the exhaled flow. One or more of these mechanisms may contribute to an increase in PEEP compared to a comparable prong with no protrusions for a given flow rate.

The outer wall 424 may act as a smooth trailing edge. The smooth curved shape of the outer wall 424 may aid in drawing the flow of gas back towards the outer surface 401 of the prong 400. The shape of the outer wall 424 shown in FIG. 15 may enable the exhaled gas to contact or flow adjacent to the outer wall 401 of the prong 400 over a greater length compared to an outer wall that is, for example, more rectangular in shape.

In the example shown in FIG. 15, the protrusions 420 when viewed in longitudinal cross-section have a profile that resembles the shape of a shark fin.

Further examples of a prong 500A,B,C according to the present disclosure are shown in FIGS. 16, 17 and 18. These examples have the same or similar features as prongs shown in other described examples, except that the protrusions 520A,B,C are of a different type having a different shape and size. Each prong 500A,B,C has an opening 505A,B,C at its distal end 502A,B,C.

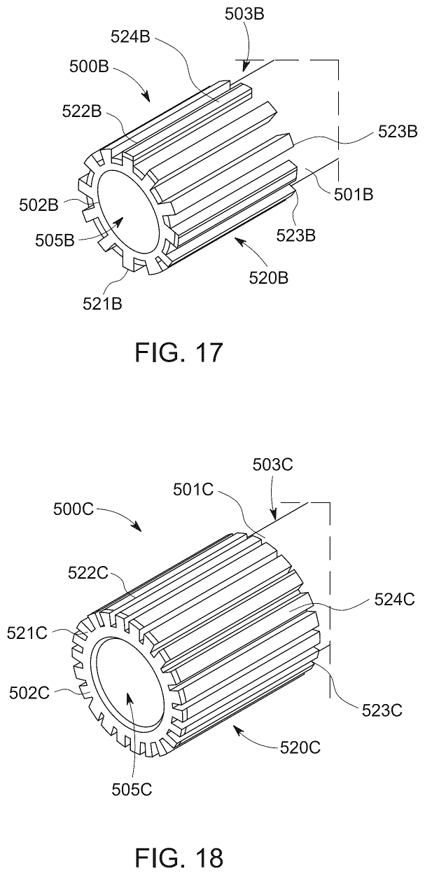

The prongs 500A,B,C shown in FIGS. 16, 17 and 18 differ from each other in the number of protrusions on each prong. Whilst these examples show specific numbers of protrusions, it is within the scope of the present disclosure that a prong may have any other number of protrusions. In the example of FIG. 16 prong 500A has four protrusions 520A, in the example of FIG. 17 prong 500B has twelve protrusions 520B and in the example of FIG. 18 prong 500C has twenty-two protrusions 520C.

Each protrusion 520A,B,C generally extends in the longitudinal direction of the prong 500A,B,C. One or more protrusion 520A,B,C may be elongate. Each protrusion 520A,B,C extends between the distal end 502A,B,C and proximal end 503A,B,C of the prong 500A,B,C. According to other examples, one or more protrusions may extend from adjacent the distal end 502A,B,C or distanced from the distal end 502A,B,C towards the proximal end 503A,B,C.

The protrusions 520A,B,C each have a first surface 521A,B,C which faces generally towards the distal end of the prong. The first surface 521A,B,C may extend substantially perpendicularly from the outer surface 501A,B,C of the prong 500A,B,C. According to other possible examples, the first surface 521A,B,C may be angled relative to the outer surface 501A,B,C of the prong 500A,B,C. Alternatively or additionally, the first surface 521A,B,C may be curved and/or rounded.

Each protrusion 520A,B,C has a pair of side walls 522A,B,C which extend longitudinally along a length of the protrusion 520A,B,C. Length of the or each protrusion may be modified to affect resistance to flow. For example, length may be increased to increase resistance to flow.

Each side wall 522A,B,C may extend substantially perpendicularly from the outer surface 501A,B,C. According to other possible examples, the side walls 522A,B,C may be angled relative to the outer surface 501A,B,C of the prong 500A,B,C. Alternatively or additionally, the side walls 522A,B,C may be curved and/or rounded.

According to some examples, the side walls 522A,B,C may extend inwardly and towards one another. The side walls 522A,B,C may meet at a point or edge. According to some examples, the side walls 522A,B,C may be angled outwardly from the outer surface 501A,B,C of the prong 500A,B,C and away from one another.

A second surface 523A is provided at the proximal end of the protrusion 520A. The second surface 523A may extend substantially perpendicularly from the outer surface 501A of the prong 500A. According to other possible examples, the second surface 523A,B,C may be angled relative to the outer surface 501A,B,C of the prong 500A,B,C. Alternatively or additionally, the second surface 523A,B,C may be curved and/or rounded.

The second surface 523A,B,C may be angled to extend from the outer surface 501A,B,C of the prong 500A,B,C substantially towards the first surface 521A,B,C. The second surface 523A,B,C may be angled to extend from the outer surface 501A,B,C of the prong 500A,B,C substantially away from the first surface 521A,B,C.

A top surface 524A,B,C is provided on the protrusion 520A,B,C. The top surface 524A,B,C spans the length of the protrusion 520A,B,C from the first surface 521A,B,C to the proximal second surface 523A,B,C and spans the width of the protrusion 520A,B,C between side walls 522A,B,C. In the examples shown, the top surface 524A,B,C is a flat planar surface that is parallel to the longitudinal direction.

In other possible examples, the top surface 524A,B,C may be angled upwardly or downwardly from the distal end to the proximal end. The first surface 521A,B,C and the second surface 523A,B,C may have different heights relative to the outer surface 501A,B,C of the prong 500A,B,C. The top surface 524A,B,C may be angled upwardly or downwardly in the circumferential direction where the side wall 522A,B,C on one side of the protrusion 520A,B,C is of a different height to the side wall 522A,B,C on the opposed side of the protrusion 520A,B,C. Alternatively or additionally, the top surface 524A,B,C may be curved and/or rounded.

As noted above, other examples are possible that have a different number of protrusions than shown in FIGS. 16, 17 and 18. In general, a prong that has more protrusions of a certain type (same width and length) may provide a higher occlusion than a prong that has less protrusions of the same type.

In use, a prong with a number of protrusions, such as shown in FIGS. 16 to 18, may provide naris occlusion of about 40-90%, or about 50-70% occlusion, and less than 100% occlusion to maintain a flow path for the exit of exhaled gas. The use of protrusions 520A, 520B, 520C of the elongate type shown in FIGS. 16, 17 and 18 may provide increased occlusion compared to a standard or comparably dimensioned prong having no protrusions. The use of protrusions 520A, 520B, 520C reduces a cross-sectional area between the outer surface 501A, 501B, 501C of the prong 500A, 500B, 500C and the inner surface of the naris, compared to an equivalently sized prong with no protrusions, which reduces the available space in which an exhaled gas may travel towards the naris exit. The use of protrusions 520A, 520B, 520C increases the cross-sectional area of the prong, compared to an equivalently sized prong with no protrusions, and therefore increases the ratio of the cross-sectional area of the prong to the cross-sectional area of the naris. The prongs 500A, 500B, 500C may provide an increased PEEP than would be provided by a comparably dimensioned prong with no protrusions for a given flow rate.

In use, exhaled gas will meet the front surface 521A, 521B, 521C of the protrusion 520A, 520B, 520C and may be redirected. This may cause regions of turbulence in the exhaled gas.

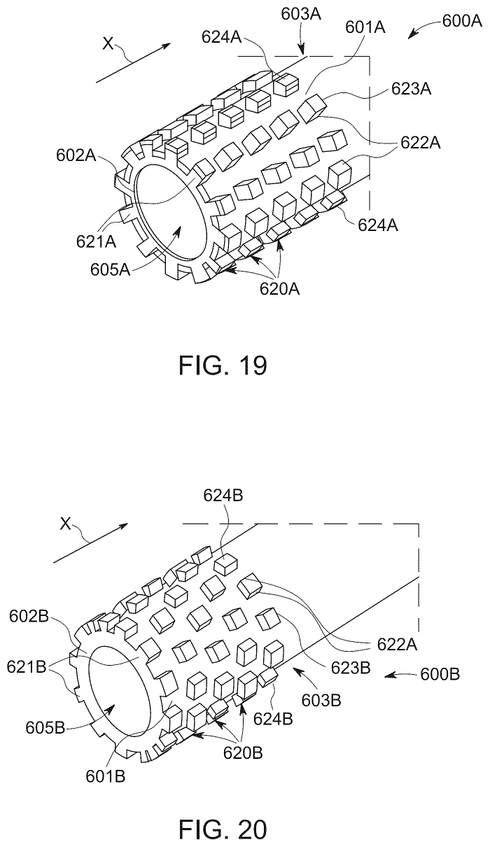

FIGS. 19 and 20 show examples of the present disclosure having prongs 600A, 600B with a plurality of protrusions 620A, 620B. These examples have the same or similar features as prongs shown in other described examples, except that the protrusions 620A,B are of a different type having a different shape and/or size. For example, the protrusions 621A, 620B in FIGS. 19 and 20 are substantially cuboid or brick-like in shape. Each prong 600A,B has an opening 605A,B at its distal and 602A,B.

The prongs 600A, 600B shown in FIGS. 19 and 20 each have a plurality of protrusions 620A, 620B. Each protrusion 620A, 620B extends from the outer surface 601A, 601B of the prong 600A, 600B and forms a substantially square prism shape. Shapes other than the brick-like or cuboid protrusions shown in FIGS. 19 and 20 are also possible within the scope of the present disclosure.

Each protrusion 620A, 620B has a first surface 621A, 621B, which faces towards distal end of the prong, generally towards the direction X of exhaled flow in use. The protrusions 620A, 620B further include side walls 622A, 622B that extend longitudinally either side of the protrusion 620A, 620B. A second surface 623A, 623B is opposed to the first surface 621A, 621B and facing towards the proximal end 603A, 603B of the prong 600A, 600B. An upper surface 624A, 624B may be provided between each of the side walls 622A, 622B, first surface 621A, 621B and second surface 623A, 623B and spaced outwardly from the outer surface 601A, 601B of the prong 600A, 600B.

According to some examples, one or more of the first surface 621A, 621B, second surface 623A, 623B, and side walls 622A, 622B of each protrusion 620A, 620B may extend substantially perpendicularly to the outer surface 601A, 601B of the prong 600A, 600B. According to some examples, the first surface 621A, 621B and second surface 623A, 623B may be substantially parallel to one another. According to some examples, side walls 622A, 622B may be substantially parallel to one another.

According to some examples, the first surface 621A, 621B, second surface 623A, 623B, and side walls 622A, 622B of protrusion 620A, 620B may be angled relative to one another such that they each extend inwardly and meet at a point. In that case, the protrusion may be substantially pyramid shaped. According to some examples, the first surface 621A, 621B, second surface 623A, 623B, and side walls 622A, 622B of protrusion 620A, 620B may be angled relative to one another such that they each extend inwardly and meet at an upper surface 624A, 624B. In that case, the protrusion may be substantially truncated pyramid shaped.

The difference between the example shown in FIG. 19 and the example shown in FIG. 20 is that in FIG. 19 the protrusions 620A are provided in circumferentially and longitudinally aligned rows. In FIG. 20, the protrusions 620B are aligned circumferentially in rows, but are staggered and offset longitudinally. In other words, the protrusions 620B in each row of FIG. 20 are staggered relative to the protrusions 620B in each adjacent row. According to some examples, the protrusions in one row may be partially offset relative to the protrusions in adjacent rows.

The example of FIG. 19 resembles the example shown in FIG. 17 but with sections of the elongate or longitudinally arranged protrusions being absent periodically along the longitudinal length. The end-on occlusion in the examples of FIGS. 19 and 17 is substantially the same. The arrangement of protrusions 620A in the example of FIG. 17 provides an interaction between the exhaled gas and the first surface 621A of each distal-most protrusion 620A. A tortuous path may be provided between protrusions 620A of each circumferential row.

Gaps between protrusions 620A may create a winding and substantially tortuous flow path rather than a flow path that can travel in only one direction along the outer surface 501B of the prong 500B, such as in the example shown in FIG. 17. This may provide a decrease in dynamic pressure and increase in static pressure of exhaled gas in use of prong 600A, compared to the prong 500B of FIG. 17 or a prong with no protrusions. The prong 600A of FIG. 19 may generate a higher PEEP than the prong 500B of FIG. 17. One reason may be that there is a higher surface area, particularly of first surfaces 621A, which contacts the flow of exhaled gas.

Staggering the protrusions 600B between adjacent rows as shown in FIG. 20 may further increase the surface area being contacted by flow of exhaled gas compared to the example of FIG. 19. As with other examples, the first surfaces 621A, 621B of the protrusions 620A, 620B reduce the available cross-sectional area between the prong and the naris through which the exhaled gas can flow compared to a prong is the same dimensions without protrusions. This arrangement of protrusions may prevent at least a portion of the flow of exhaled gas from exiting the naris in a substantially straight line. When the flow of exhaled gas meets a first surface 621B, there is a redirection of exhaled gas, a mixing of the gas and/or an increased turbulence in the flow. This may result in a further reduction in dynamic pressure and an increase in static pressure. The prong 600B of FIG. 20 may provide an increase in PEEP compared to the prong 600A of FIG. 19 due to the staggered arrangement of protrusions 600B increasing interactions between portions of the flow of exhaled gas and the first surfaces 621B.

Increasing the number of protrusions 600A, 600B in one or each circumferential row may increase PEEP compared to examples with fewer protrusions 600A, 600B. The optimum number of protrusions in each row to provide a desired PEEP may be dependent on one or more dimensions of the protrusions, the shape of the protrusions and the percentage of occlusion of a given naris by the prong.

The first surface 621A, 621B and second surface 623A, 623B of the protrusions 600A, 600B may also be considered as side walls of the protrusions 600A, 600B. For example, the protrusions may have four side walls and a rectangular or square cross-sectional shape, as shown in FIGS. 19 and 20. The side walls of each protrusion may each be equally sized and shaped. The protrusions may have any desired number of side walls. The protrusion may have a triangular cross-section and three side walls, for example.

The protrusions 620A, 620B shown in FIGS. 19 and 20 could, according to some examples, be replaced with protrusions of a similar shape which are angled relative to those shown in the Figures. For example, instead of having a first surface that faces generally toward the distal end of the prong, one or more protrusion may have an edge formed by a side walls meeting, or a corner between side walls. Similarly, a proximal-most point on the or each protrusion may be a corner between different side walls of the protrusion.

In some examples, the protrusions 620A, 620B may be elongate in the longitudinal direction of the prong. The protrusions may be provided in rows circumferentially about the prong. The protrusions may be provided uniformly or non-uniformly over the surface of the prong.

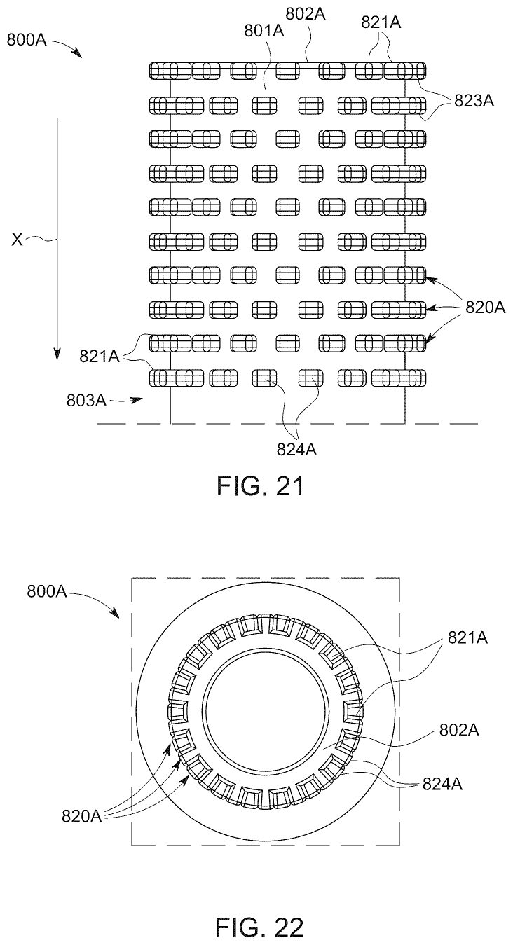

FIGS. 21 to 26 show further examples of a prong 800A, 800B, 800C having protrusions 820A, 820B, 820C according to the present disclosure. These examples have the same or similar features as the prongs shown in other described examples, except that the protrusions 820A,B,C may be of a different type having a different shape and/or size.

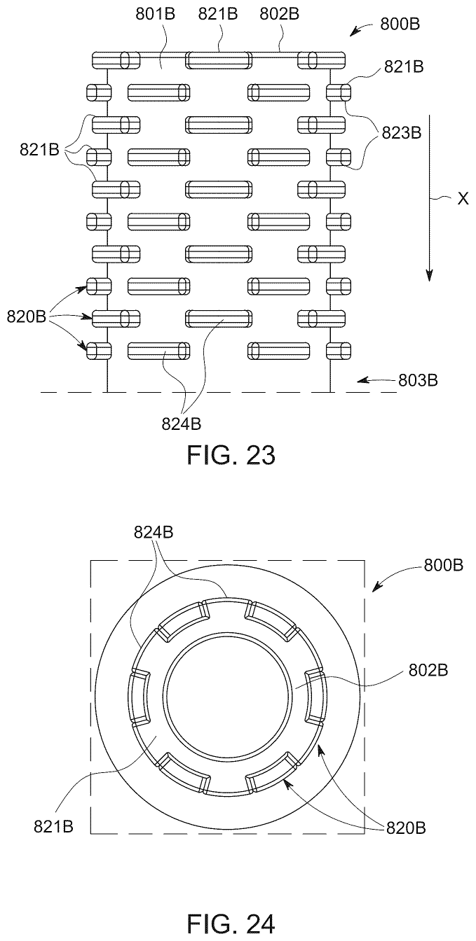

Each protrusion 820A, 820B, 820C extends from the outer surface 801A, 801B, 801C of the prong. The protrusions 820A, 820B, 820C of the examples of FIGS. 21 to 26 have a greater extent in the circumferential direction around the prong 800A, 800B, 800C than in the longitudinal direction. The protrusions 820A in FIGS. 21, 22 are only slightly more elongate in the circumferential direction than in the longitudinal direction. The protrusions 920B, 820C in FIGS. 23 to 26 particularly, are elongate in the circumferential direction.

The protrusions may be substantially rectangular prism shape and/or may have one or more curved edges and/or curved corners. As shown in FIGS. 21 to 26, each protrusion 820A, 820B, 820C has a first surface 821A, 821B, 821C, facing generally towards the distal open end of the prong. In use, first surface 821A, 821B, 821C may face generally towards direction X of exhaled flow. The protrusions 820A, 820B, 820C include side walls 822A, 822B, 822C that extend longitudinally either side of the protrusion 820A, 820B, 820C. A second surface 823A, 823B, 823C generally opposes the first surface 821A, 821B, 821C and towards the proximal end 803A, 803B, 803C of the prong 800A, 800B, 800C. An upper surface 824A, 824B, 824C is provided on the protrusions 820A, 820B, 820C between each of the side walls 822A, 822B, 822C, first surface 821A, 821B, 821C and second surface 823A, 823B, 823C and spaced outwardly from the outer surface 801A, 801B, 801C of the prong 800A, 800B, 800C.

According to some examples, one or more of the first surface 821A, 821B, 821C, second surface 823A, 823B, 823C, and side walls 822A, 822B, 822C of each protrusion 820A, 820B, 820C may extend substantially perpendicularly to the outer surface 801A, 801B, 801C of the prong 800A, 800B, 800C. According to some examples, the first surface 821A, 821B, 821C and second surface 823A, 823B, 823C of a protrusion 820A, 820B, 820C may be substantially parallel to one another.

According to some examples, side walls 822A, 822B, 822C of a protrusion 820A, 820B, 820C may be substantially parallel to one another.

According to some examples, any one or more of the first surface 821A, 821B, 821C, second surface 823A, 823B, 823C, and side walls 822A, 822B, 822C of protrusion 820A, 820B, 820C may be angled relative to one another such that they extend inwardly. According to some examples, the first surface 821A, 821B, 821C, second surface 823A, 823B, 823C, and side walls 822A, 822B, 822C of protrusion 820A, 820B, 820C may be angled relative to one another such that they each extend inwardly and meet at a point. In that case, the protrusion may be a pyramid-like structure.

According to some examples, the first surface 821A, 821B, 821C, second surface 823A, 823B, 823C, and side walls 822A, 822B, 822C of protrusion 820A, 820B, 820C may be angled relative to one another such that they each extend inwardly and meet at an upper surface 824A, 824B, 824C. In that case, the protrusion may be a truncated pyramid-like structure.

The upper surface 824A, 824B, 824C may be substantially flat or planar or may be curved or rounded. Any one or more of the first surface 821A, 821B, 821C, second surface 823A, 823B, 823C, and side walls 822A, 822B, 822C of protrusion 820A, 820B, 820C may be substantially flat or planar or may be curved or rounded.

The difference between the examples of FIGS. 21 to 26 and the example shown in FIG. 20 is that the protrusions 800A, 800B, 800C in FIGS. 21 to 26 are elongate in the circumferential direction compared to their extent in the longitudinal direction. The presence of a widened first surface 821A, 821B, 821C, assuming the height of the protrusion 820A, 820B, 820C remains the same, may increase the surface area with which exhaled flow direction X directly interacts. An increase in the circumferential extent of the first surface 821A, 821B, 821C of the protrusion 820A, 820B, 820C may lead to an increase in PEEP. An increase in the circumferential extent of the first surface 821A, 821B, 821C of the protrusion 820A, 820B, 820C may increase the occlusion of the naris at that location.

The prong 800A shown in FIGS. 21, 22 has ten rows of protrusions 820A. Each circumferential row in this example has eighteen protrusions 820A.

The prong 800B shown in FIGS. 23, 24 has ten rows of protrusions 820B. Each circumferential row in this example has six protrusions 820B.

The prong 800C shown in FIGS. 25, 26 has ten rows of protrusions 820C. Each circumferential row in this example has two protrusions 820C. Prongs having different numbers of circumferential rows and/or different numbers of protrusions in each row than those shown in FIGS. 21 to 26 are also possible within the present disclosure.

A prong according to the present disclosure may comprise any of one, two, three, four, five, six, seven, eight, nine, ten, eleven, twelve, thirteen, fourteen, fifteen, sixteen, seventeen, eighteen, nineteen, twenty or more than twenty rows of protrusions. The prong may comprise any of: one to twenty, one to fifty, two to twenty, five to twenty, ten to twenty, five to fifteen, or five to ten rows or protrusions.

Each row may comprise any desired number of protrusions. For example, each row may comprise any one of: one to fifty, one to thirty, one to twenty, one to ten, five to fifty, five to twenty, five to fifteen, five to ten, ten to fifty, ten to thirty, or ten to twenty protrusions. Each row may comprise the same number of protrusions as each other row of protrusions. At least one row of protrusions may comprise a different number of protrusions to at least one other row of protrusions.

In the examples shown in FIGS. 21 to 26, the gaps in the circumferential direction between adjacent protrusions 820A, 820B, 820C in a row are substantially equal to the circumferential length of each protrusion 820A, 820B, 820C. Gaps between the protrusions 820A, 820B, 820C may be larger than the circumferential length of each protrusion 820A, 820B, 820C. Gaps between the protrusions 820A, 820B, 820C may be smaller than the circumferential length of each protrusion 820A, 820B, 820C.

The examples shown in FIGS. 21 to 26 have protrusions 820A, 820B, 820C that are arranged on the outer surface 801A, 801B, 801C of the prong 800A, 800B, 800C in a staggered arrangement. Protrusions 820A, 820B, 820C of one row are offset relative to the protrusions 820A, 820B, 820C of each adjacent row. Other arrangements of protrusions of this type are also possible within the scope of the present disclosure. For example, the protrusions of each row may be aligned longitudinally with adjacent rows or the protrusions could be arranged randomly.

The wider the protrusion becomes in the circumferential direction of the prong, the closer the protrusion will come to a full ring. A protrusion that is elongate over the entire circumference of the prong may increase PEEP in use. However, a protrusion that encompasses the entire circumference will increase the possibility that the naris will be sealed when the prong is inserted. Sealing the naris is to be avoided, particularly in high flow or other flow-controlled therapy. Therefore, it may be preferred that the protrusions are not so wide that they traverse the entire circumference of the prong. Instead, at least one gap in the circumference of each protrusion or each circumferential row of protrusions may be present to reduce the possibility of the prong sealing a naris. The gaps between protrusions in a row may assist to provide a safe passageway for exhaled gas to exit the naris.

The function of the first surfaces 821A, 821B, 821C of the protrusions 820A, 820B, 820C in the examples of FIGS. 21 to 26 is similar to that of the first surfaces 621B of the protrusions 620B of the example of FIG. 20. When a flow of exhaled gas contacts the first surface 821A, 821B, 821C, the gas may be redirected. The gas may mix within the flow creating an increase of turbulence. This may lead to an increase in PEEP in the patient airway compared to a comparable prong with no protrusions present for a given flow rate. Staggering the protrusions 820A, 820B, 820C between rows, as shown in FIGS. 21 to 26, may further increase the PEEP. The exhaled gas flow may undergo sudden narrowing and expansion in the flow path between the protrusions 820A, 820B, 820C.

The example of FIGS. 25, 26 may provide a more tortuous path for a flow of exhaled gas over the outer surface 801C of the prong 800C compared to the examples shown in FIGS. 21 to 24. The reason for this is that the protrusions 820C and their first surfaces 821C in FIGS. 25, 26 are wider and the flow path required for gas to navigate around the protrusions 820C may be longer than required to navigate around the protrusions 820A, 820B of FIGS. 21 to 24. The PEEP generated at least in part by the prongs 800A,B,C may be different from one another. The actual PEEP provided may be determined by the dimensions and/or shape of the protrusions 820A,B,C, the occlusion of a particular naris with the prong 800A,B,C, the average flow rate of exhaled gas and/or any other parameter which affects the PEEP.

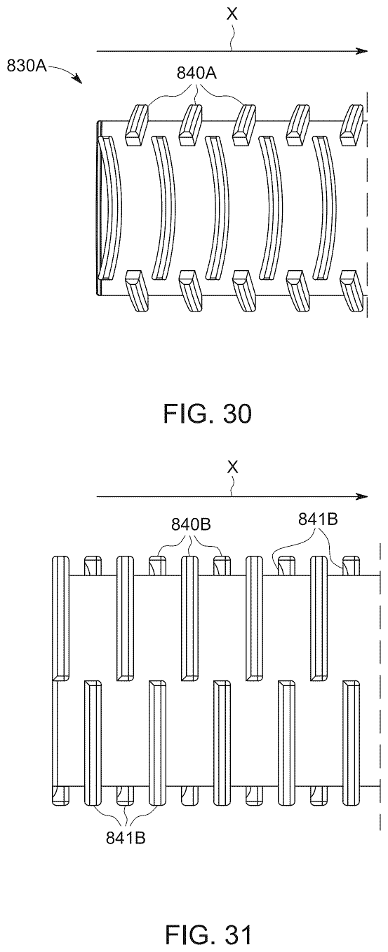

Alterations to the configuration of the protrusions 820A, 820B, 820C of the examples of FIGS. 21 to 26 are possible. For example, FIGS. 30 to 32 show example configurations of protrusions that are elongate in, generally, the circumferential direction. FIG. 30 shows an example where each protrusion 840A is curved.

FIG. 31 shows an example where each protrusion 840B has a concave face. In the example shown, the concave face is on the first surface of the protrusion which faces substantially towards the distal end of the prong. This feature of a concave face may be present on any other protrusion surface disclosed herein, such as side walls, distal or proximal surfaces or outer surfaces for example.

Alternatively or additionally, any protrusion disclosed herein may include a convex face. The example of FIG. 30 could include protrusions having a concave face. The inclusion of at least one concave or convex surface on at least one protrusion may be included on a prong having any number of protrusions.

Each protrusion in these examples has a circumferential width. In the examples of FIGS. 30 and 31, the circumferential gap between the protrusions in each row is substantially the same dimension as the circumferential width of the protrusions 840A, 840B. The widths of each protrusion and the gaps between each protrusion in a row may be varied and not the same as one another.

FIG. 30 shows an example where the protrusions 840A on the prong 830A are curved towards the proximal end of the prong between each end of the protrusions 840A. The protrusions 840A are curved in a concave manner with respect to the distal end of the prong 830A. Each circumferential end of the elongate curved protrusions 840A in FIG. 30 are positioned towards the distal end compared to a centre of the protrusions 840A.

FIG. 31 shows an example of protrusions 840B that have a concave indentation on the first surface 841B. Similarly to other described examples, the first surface 841B in use 840B faces towards the exhaled flow direction, in other words, towards the distal end of the prong 830B. According to other possible examples, one or more other faces of the or each protrusion, such as a side wall, second or trailing surface and/or an outer surface, may additionally or alternatively be curved or have a concave or convex indentation.

A height h, shown in FIG. 27, of circumferentially elongate protrusions may be increased and may provide an increase in naris occlusion compared to a prong of the same dimensions without protrusions. This increase in occlusion may lead to an increase in PEEP in use due at least in part to the increased resistance encountered by the exhaled gas. A peak improvement in PEEP experienced by patient, assuming comparison between the same type of protrusions, may be achieved at about 50-70%, about 70% occlusion or above 70% (but less than 100%) occlusion. For occlusions that are not distinctly different to the profile of a standard prong, such as where the height h is small, there may be a smaller increase in the PEEP in use compared to a comparable prong with no protrusions.

FIGS. 27 to 29 show end views of some possible arrangements and alignments of protrusions 870A, 870B, 870C, 871A, 871B, 871C on a prong 850A, 850B, 850C. Here there are shown two protrusions in each of two adjacent rows. In these examples, the height h of each protrusion is the same. However, it may be that the protrusions of any disclosed examples have variable heights. The height of each protrusion may be selected to provide a desired naris occlusion.

In the example of FIG. 27, the protrusions 870A of a first row are staggered or arranged offset from the protrusions 871A of the second row. Gaps between the protrusions 870A, 871A in each of these rows is larger than the width of the protrusions such that there is no overlap between the protrusions 870A of the first row and the protrusions 871A of the second row. In this example, gaps 880A are provided where no protrusions 870A, 871A in either of the first or second rows are present.

In the example of FIG. 28, the protrusions 870B of the first row are staggered from the protrusions 871B of the second row. In this example, gaps between protrusions 870B, 871B are the same size as the width of the protrusions 870B, 871B. Edges of each protrusion 870B in the first row are aligned with edges of protrusions 871B in the second row. For configurations where there are more than two rows of protrusions, there may be a similar alignment between the ends/edges of the protrusions of adjacent rows.

In the example of FIG. 29, protrusions 870C of the first row are partially staggered from protrusions 871C of the second row. In this example, gaps between protrusions 870C, 871C are the same size as the width of the protrusions 870C, 871C. The partial staggering of rows provides sections of overlap 890C where protrusions 870C of the first row overlap with protrusions 871C of the adjacent row. In this example gaps 880C are provided in two locations where no protrusions 870C, 871C in either of the first or second rows are present. According to some configurations there may be a third row, fourth row, and so on for the number of rows of protrusions. In those cases, there may be an overlap between protrusions of adjacent rows.

Having an overlap of protrusions between rows, such as shown in FIG. 29, may increase patient pressure, for example, PEEP compared to configurations where no overlap between rows is present. PEEP may differ depending on the amount of overlap between protrusions of adjacent rows. In some cases, there may be an optimum overlap amount between protrusions of adjacent rows. Increasing the amount of overlap of protrusions between adjacent rows above the optimum amount may decrease PEEP.

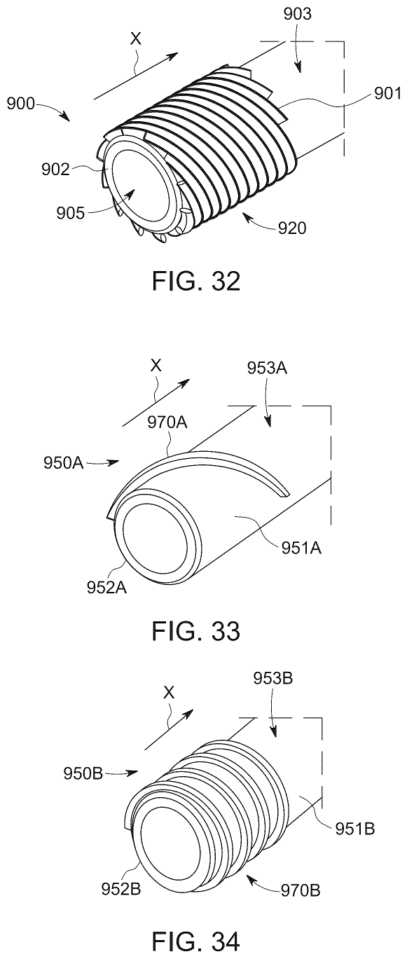

Further examples of prongs having one or more protrusions on their outer surface are shown in FIGS. 32 to 36. Each of these examples utilises helically arranged protrusions about the outer surface of the prong.

FIG. 32 shows an example of a protrusion 900 with a plurality of protrusions 920 over its outer surface 901. This example has the same or similar features as prongs shown in other described examples, except that the protrusions 920 are of a different type having a different shape and/or size. The prong 900 has an opening 905 at its distal end 902.

In the example shown in FIG. 32, the prong 900 includes twelve protrusions 920. Other examples having a similar type but different number of protrusions 920 are also possible within the scope of the present disclosure. For example, a prong 900 may include one, two, three, four, five, six, seven, eight, nine, ten, eleven, twelve, thirteen, fourteen, fifteen, sixteen, seventeen, eighteen, nineteen, twenty or more than twenty protrusions.

Each protrusion 920 shown in FIG. 32 is in the form of a wall that extends helically around the prong 900 between the distal end 902 and the proximal end 903. This example may be considered to be similar to elongate protrusions as described above except that the elongate protrusions in this example are angled relative to the length of the prong 900 such that they extend helically. Each protrusion 920 extends outwardly from the outer surface 901 of the prong 900. Use of the prong 900 with helical protrusions 920 of FIG. 32 may provide increased PEEP to a patient compared to the use of a standard prong with no protrusions. Increasing the number of helical protrusions may increase PEEP.

Helical flow paths for exhaled gas are provided between adjacent pairs of protrusions 920. These gas flow paths follow the same helical line as the protrusions 920. In use, the helical protrusions 920 may provide the exhaled gas with a winding and/or tortuous path between the distal end 902 and proximal end 903 of the prong 900. The winding nature of the flow path may lead to an increased length of the exit path compared to at least some other examples described herein.

FIGS. 33, 34, 35, 36 show examples of a prong 950A, 950B, 950C, 950D having a single helical protrusion 970A, 970B, 970C, 970D. Similarly to FIG. 32, protrusions 970A, 970B, 970C, 970D extend helically around the outer surface 951A, 951B, 951C, 951D of the prong 950A, 950B, 950C, 950D between the distal end 952A, 952B, 952C, 952D and the proximal end 953A, 953B, 953C, 953D.

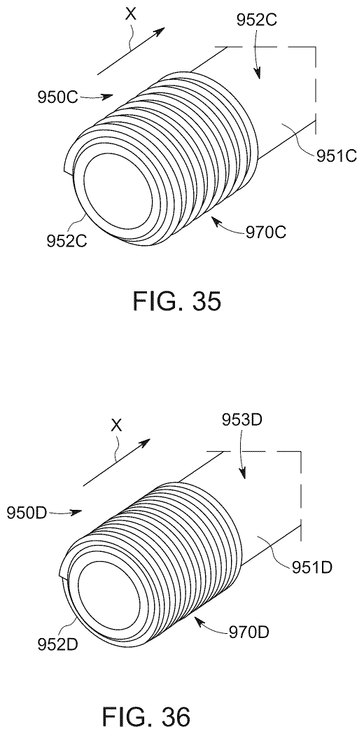

The prong 950A shown in FIG. 33 has a single protrusion 970A that extends helically about the prong 950A by one half of a revolution. The term revolution in this context is used to refer to the number of times the protrusion extends around the circumference of the prong. The prong 950B shown in FIG. 34 has a single protrusion 970B that extends helically about the prong 950B by four revolutions. The prong 950C shown in FIG. 35 has a single protrusion 970C that extends helically about the prong 950C by eight revolutions. The prong 950D shown in FIG. 37 has a single protrusion 970D that extends helically about the prong 950D by sixteen revolutions.

The examples shown in FIGS. 33 to 36 may provide a winding and/or tortuous path of exit along each prong for at least a portion of the exhaled gas. The winding flow path is provided by the helical revolution(s) of the single protrusion.

Each helical protrusion may have a pitch or angle to the longitudinal direction of about 20 to 80 degrees or 30 to 70 degrees.

The helical protrusions in the examples of FIGS. 32 to 36 each extend outwardly from the respective outer surface of the prong. The protrusion(s) reduce the available cross-sectional area between the outer surface of the prong and the inner surface of the naris compared to a prong of the same dimensions with no protrusions. The protrusion(s) may increase resistance to flow of exhaled gas compared to a standard prong with no protrusions. The protrusion(s) may reduce the dynamic pressure and may increase static pressure. The result may be an increase in PEEP.

In the examples discussed herein, the patient interfaces comprise a pair of prongs. According to some configurations a patient interface may comprise a single prong of any type disclosed herein. A patient interface may include one prong having at least one protrusion on its outer surface and one prong with no protrusions on its outer surface.

Where a patient interface, including but not limited to a nasal cannula, has at least two prongs, the prongs may have the same cross-sectional area as one another. The prongs may have a cross-sectional area that differs between two prongs.

As described in PCT publications WO2015020540A1 and WO2022229909A1 of the present applicant the prongs of a patient interface may be asymmetric. The content of each of these PCT publications is included herein in its entirety. In general, the asymmetry may mean that one prong is larger in at least one dimension than the other prong. One prong may differ in shape to the other prong.

One prong may have a greater inner (lumen) cross-sectional area than the other prong. One prong may have a greater outer cross-sectional are than the other prong. One prong may be more flexible than the other prong. One prong may be longer than the other prong. One prong may have an increased inner or outer circumference than the other prong. One prong may have a larger wall thickness than the other prong.

An asymmetric prong design, where the two prongs are sized and/or shaped differently to one another, including where the protrusions of one prong differ to that of the other, or where one prong includes at least one protrusion and the other prong has no protrusions, as described below, may have one or more of the advantages of:
improving dead space clearance in patient upper airway;
contributing to increased patient airway pressure, such as PEEP. This may be provided by the prongs in combination providing a larger combined cross-sectional area where one prong is enlarged, for example, by arrangement of protrusions, compared to the other. A larger combined cross-sectional area may occlude a larger cross-sectional area of the nares, contributing to an increased patient pressure for a given flow rate; and
may mitigate risks associated with completely sealing patient airways.

A combined cross-sectional area of two prongs where one prong is sized larger than the other, may be larger than the combined cross-sectional area for two prongs of the same size dimensions. The combined diameter of the two prongs where one is larger than the other may be the same as the combined diameter of the two prongs that are the same size.

In some configurations a nasal interface of the present disclosure comprises a first prong and a second prong that are asymmetrical to each other. The first prong may refer to either the left or right prong and accordingly the second prong would refer to either the right or left prong.

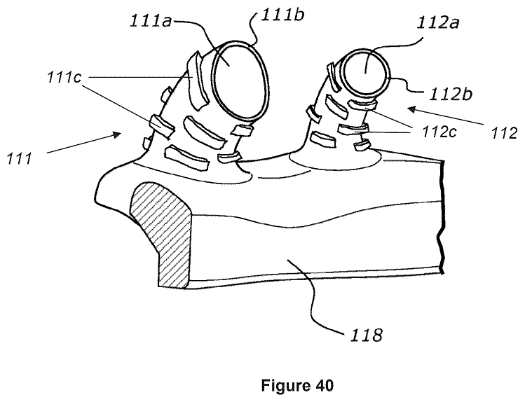

Referring to FIG. 40, the first prong 111 has a first distal end 111b adjacent the first opening 111a. The second prong 112 has a second distal end 112b adjacent the second opening 112a. The first prong 111 has protrusions 111c on its outer surface. The second prong 112 has protrusions 112c on its outer surface. The first prong 111 is larger in cross-sectional area than the second prong 112.

In the example shown in FIG. 40, the inner passage or lumen of the first prong 111 is larger than the inner passage or lumen of the second prong 112. However, the inner passage or lumen of the first prong 111 may be the same or similar to the inner passage or lumen of the second prong 112. Where the inner passage or lumen of each prong is the same or similar, asymmetry of prongs may be achieved by different

The protrusions 111c on the first prong 111 are larger than the protrusions 112c on the second prong 112. The protrusions 111c may be larger than the protrusions 112c in any dimension such as any one or more of width, height, length. The protrusions 111c may differ from the protrusions 112c in shape or type of protrusion. According to other examples, one prong may not include any protrusions and the other prong may include at least one protrusion.

In the example shown in FIG. 40 the protrusions 111c, 112c are circumferentially elongate protrusions. The prongs 111, 112 may include protrusions of any size or shape possible. For example. The prongs 111, 112 may include any of the protrusions as substantially described herein in relation to one or more of FIGS. 8 to 36. The protrusions 111c of the first prong 111 may be different in size, shape or type to the protrusions 112c of the second prong 112.

According to the present disclosure, an open system is maintained by ensuring there is a gap between the outer surface of each prong and the inner surface of each naris. The open system may be maintained even where there is an asymmetry between the two prongs. The open system means there is a gap between the prong(s) and the inner surface of the nares. This ensures there is a safe flow path for exhaled gases to be expelled from the nares.

Where there is an asymmetry between the protrusion(s) on a first prong and the protrusion(s) on a second prong it may lead to one naris being occluded to a greater extent than the other naris. This may also lead to an increased clearance surrounding one prong in the naris compared to the other prong in the naris. In general, the prong that provides a greater clearance will not be the prong that provides increased occlusion. A similar asymmetry may be provided where one prong includes at least one protrusion and the other prong does not have any protrusions.

Asymmetry between the first prong and the second prong may be provided through a difference or variation in the protrusions on the outer surface of each prong in various ways. Potential features that may differ between the protrusions on one prong compared to the other prong are as follows and the features of each prong may be selected from any one or combination of these.

Each prong may have protrusions of the same type. Each prong may have protrusions that differ in some aspect to the protrusions on the other prong. For example, the protrusions of one prong may be of a different type to the protrusions of the other prong. The protrusions of one prong may have a larger height than the protrusions of the other prong. The circumferential cross-sectional area of the protrusions of one prong may be larger than the circumferential cross-sectional area of the protrusions of the other prong. The shape of the protrusions on one prong may be different to the shape of the protrusions on the other prong.

According to some examples, the dimensions of the protrusions on each of the prongs is substantially the same as one another. According to some examples, the protrusions of one prong are larger than the protrusions of the other prong. Where one prong is larger than the other prong, the larger prong may include larger protrusions than the other prong. The size of the protrusions relative to the prong on which the protrusions are located may be consistent between prongs. In other words the ratio of prong size (e.g. one or more of width, length, circumference, wall thickness, cross-sectional area) to the size of the protrusions (e.g. height, width, length, cross-sectional area) is substantially the same in each of the prongs.

Each prong may include the same number of protrusions. Each prong may include a different number of protrusions. The number of protrusions on each prong may be relative to the size of that prong compared to the other prong. One prong may have a larger number of protrusions than the other prong. One prong may include at least one protrusion and the other prong may include no protrusions. One prong may include a plurality of protrusions and the other prong may include no protrusions.

Where at least one prong has protrusions arranged in rows, the number of protrusions per row may be larger on one prong compared to the other prong. The number of protrusions per row may be the same in each prong. The number of rows of protrusions may be greater on one prong than the other prong. The number of rows of protrusions on each prong may be the same. The alignment of protrusions between adjacent rows may vary between one prong and the other prong. The alignment of protrusions between adjacent rows of protrusions on each prong may be the same.

One prong may have protrusions in rows that are in alignment with protrusions in and adjacent row. One prong may have protrusions in rows that are offset or staggered with the protrusions in an adjacent row. The degree or amount of offset or staggering of protrusions from one row to an adjacent row may differ between one prong and the other. One prong may include protrusions that are aligned in row and the other prong may include protrusions that are not aligned in rows. One prong may include protrusions that are equally or uniformly spaced from adjacent protrusions and the other prong may include protrusions that are unevenly spaced from one another or that do not have a uniform spacing.

By providing protrusions asymmetrically between the first prong and the second prong a difference in occlusion between the nares may be provided.

The safe flow path may be enhanced via an asymmetry in size between the prongs. One prong that is smaller may have an inherent gap between its outer surface and the inner surface of the naris which may further mitigate any risk of fully occluding the nares. If the prong with the larger cross section (at the protrusion) has a gap—either inherently or via a ‘safe’ gas flow path, it may be assured that the smaller prong does not occlude the naris, so there will always be an inherent gap in one or both nares.

According to the present disclosure a number of examples, configurations and/or embodiments are disclosed. The skilled person will understand that any one of more of the features of one example or configuration described herein may be combined with any one or more feature of another example or configuration described herein. Features of a plurality of different examples or configurations may be combined together in any manner selected by a skilled person which may achieve a desired outcome, product or beneficial result. For example, any one or more of the features of any of FIGS. 1 to 42 may be combined with any one or more features of any one or more of the other Figures. Any one or more features or elements of one or more of the examples or configurations herein may be removed from that example or configuration where said feature(s) are non-essential to the achievement of a desired outcome, product or beneficial effect.

While the above description includes a limited number of examples or configurations, it will be appreciated by those skilled in the art that many alternative, modifications and variations in light of the foregoing description are possible. Accordingly, the present disclosure is intended to embrace all such alternative, modifications and variations as may fall within the spirit and scope of the present disclosure.

Any reference to or discussion of any document, act or item of knowledge in this specification is included solely for the purpose of providing a context for the present invention. It is not suggested or represented that any of these matters or any combination thereof formed at the priority date part of the common general knowledge, or was known to be relevant to an attempt to solve any problem with which this specification is concerned.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

1.-96. (canceled)

97. A nasal cannula comprising a prong configured to be inserted into a naris of a patient, wherein the prong comprises:
a proximal end having an opening configured to deliver a gas into the naris;
a distal end configured to be in fluid communication with a gas source;
an inner surface defining a passage between the proximal end and the distal end;
an outer surface; and
at least one protrusion on the outer surface of the prong, wherein the at least one protrusion is elongate in a circumferential direction and provides an increased resistance to a flow of exhaled gas along the outer surface of the prong.

98. The nasal cannula of claim 97, wherein a gap is maintained in the at least one protrusion circumferentially to prevent the naris of the patient from being occluded and ensure a flow path for exhaled gases.

99. The nasal cannula of claim 97, wherein the prong comprises a plurality of protrusions and the plurality of protrusions are arranged in rows, wherein the rows are arranged substantially circumferentially around the outer surface of the prong.

100. The nasal cannula of claim 99, wherein the plurality of protrusions of one of the rows is aligned in a longitudinal direction with the plurality of protrusions in an adjacent row.

101. The nasal cannula of claim 97, wherein the at least one protrusion comprises an elongate wall extending from the outer surface of the prong and extending a partial distance around a circumference of the prong.

102. The nasal cannula of claim 98, wherein the at least one protrusion comprises a plurality of protrusions arranged in at least one row about a circumference of the prong, the gap being provided in the circumferential direction between adjacent protrusions of the at least one row, wherein the gap is substantially equal to, larger than or smaller than a circumferential length of each protrusion.

103. The nasal cannula of claim 97, wherein the at least one protrusion is substantially prism shaped comprising curved edges and/or corners.

104. The nasal cannula of claim 97, wherein one or more of a first surface, a second surface, and a side wall of the at least one protrusion extends substantially perpendicularly to the outer surface of the prong.

105. The nasal cannula of claim 97, comprising:
a body comprising a central section; and
a pair of side arms on either side of the body,
wherein the prong extends from the central section of the body.

106. The nasal cannula of claim 97, comprising a patient securement assembly comprising one or more facial pads located on side arms.

107. The nasal cannula of claim 97, wherein the prong comprises a cross-section that is circular or substantially elliptical or oval.

108. The nasal cannula of claim 97, wherein the prong tapers inwards along a length of the prong.

109. The nasal cannula of claim 97, wherein the prong has a curved shape between the proximal end and the distal end.

110. The nasal cannula of claim 97, wherein the prong is configured to be non-sealing with a nare of a patient.

111. The nasal cannula of claim 97, comprising:
a pair of prongs and a manifold in fluid communication with the pair of prongs, wherein the pair of prongs is configured to receive gases from the gas source via a common conduit, or
a pair of prongs, wherein each prong is configured to receive gas from separate conduits.
